(19) United States
(12) Patent Application Publication
(10) Pub. No.: US 2023/0338432 A1
(43) Pub. Date: Oct. 26, 2023

(54) METHODS AND COMPOSITIONS FOR TREATING VIRAL INFECTIONS AND SEQUELAE THEREOF

(71) Applicant: PLURISTEM LTD., Haifa (IL)

(72) Inventors: Yaacob YANAY, Shimshit (IL); Rachel OFIR, Adi (IL); Marc TRITEL, Bet Shemesh (IL)

(73) Assignee: PLURISTEM LTD., Haifa (IL)

(21) Appl. No.: 17/787,616

(22) PCT Filed: Mar. 11, 2021
(86) PCT No.: PCT/IL2021/050268

§ 371 Date: Jun. 21, 2022

Related U.S. Application Data
(60) Provisional application No. 62/988,815, filed on Mar. 12, 2020.
(60) Provisional application No. 63/006,117, filed on Apr. 7, 2020.
(60) Provisional application No. 63/049,144, filed on Jul. 8, 2020.

(30) Foreign Application Priority Data
May 14, 2020 (IL) 274697

(51) Int. Cl. A61K 35/50; A61K 39/00; A61P 31/14

(52) U.S. Cl. CPC A61K 35/50; A61K 39/461; A61K 39/4622; A61K 39/464429; A61K 39/464411; A61K 39/464412; A61K 39/4634; A61K 39/4643; A61P 31/14

(57) ABSTRACT

Disclosed herein are methods and compositions comprising placental adherent stromal cells for treating viral infections and sequelae thereof.

### FIELD

Disclosed herein are methods and compositions for treating viral infections and sequelae thereof.

### BACKGROUND

On 31 Dec. 2019, WHO was informed of a cluster of cases of pneumonia of unknown cause detected in Wurlehan City, Hubei Province of China. The causative agent was found to be a coronavirus, given the name SARS-CoV-2 (severe acute respiratory syndrome coronavirus 2), and the disease named “COVID-19” (Corona Virus Disease, 2019). The virus was found to confer high risk for various lung morbidities, and was associated with ICU admission and high mortality. There are now over 100,000,000 confirmed cases and 2.5 million deaths from COVID-19.

In most patients, SARS-CoV-2 infections are either asymptomatic or present as cases resembling the seasonal flu or those with a mild form of pneumonia. However, some patients exhibit severe disease and develop acute respiratory distress syndrome (ARDS), a clinical phenomenon marked by development of bilateral infiltrates and hypoxemia, defined as a decrease in the ratio of the partial pressure of arterial oxygen to the fraction of inspired oxygen (PaO₂/FiO₂). Many COVID-19 patients who develop ARDS require invasive mechanical ventilation for 10-14 days, and up-to 80% of those patients ultimately succumb to the disease.

Severely affected patients can also develop a syndrome of dysregulated and systemic immune overactivation, often termed “cytokine storm” or “hyperinflammatory syndrome”. Cytokine storm, associated with an excessive production of proinflammatory cytokines and considered to be one of the major causes of vascular hyperpermeability that worsens the symptoms of ARDS, may lead to multisystem organ failure and mortality.

SIRS is a dangerous, potential inflammatory consequence of infection. A patient is considered to have SIRS if s/he meets at least 2 of the following criteria: (a) a body temperature of greater than 38 or less than 36 deg. C.; (b) a heart rate of greater than 90; (c) a respiratory rate of greater than 20 and/or PaCO₂ less than 32 mg Hg; and (d) WBC greater than 12,000/mm³ or less than 4,000/mm^{3,} or greater than 10% bands. Sepsis is defined as SIRS plus a suspected or present source of infection. Severe sepsis is indicated by lactic acidosis, SBP less than 90, or an SBP drop greater than 40 mg Hg of normal. Septic shock is indicated by severe sepsis with hypotension, despite adequate fluid resuscitation. Multiple organ dysfunction syndrome is indicated by evidence of at least 2 organs failing. All of these conditions can be sequelae of viral infection and/or virally-induced pneumonia.

Kawasaki disease (or mucocutaneous lymph node syndrome) is also suspected to be a sequela of COVID-19 and other viral infections. Kawasaki disease is characterized by swelling and/or inflammation in the walls of medium-sized arteries throughout the body. It primarily affects children. The inflammation tends to affect the coronary arteries, which supply blood to the heart muscle. Pediatric multisystem inflammatory syndrome is a syndrome characterized by persistent fever, inflammation (neutrophilia, elevated C-Reactive Protein [CRP] and lymphopaenia) and evidence of single or multi-organ dysfunction (shock, cardiac, respiratory, renal, gastrointestinal or neurological disorder), in some cases with additional features (Guidance: Paediatric multisystem inflammatory syndrome temporally associated with COVID-19, from the Royal College of Paediatrics and Child Health).

### SUMMARY

Provided herein are methods and compositions for treating and preventing acute kidney injury (AKI) and/or deterioration of patients with viral infections and complications of viral infections, comprising administration of placental adherent stromal cells (ASC), and conditioned media (CM) thereof. Such patients may be at risk, of e.g., Acute Respiratory Distress Syndrome (ARDS), sepsis, pulmonary hypertension, lung fibrosis, Kawasaki disease, pediatric multisystem inflammatory syndrome, AKI, and gastrointestinal injury.

Conditioned medi[a]/[um]/CM, as used herein, refers to a growth medium that has been used to incubate a cell culture. The present disclosure is not intended to be limited to particular medium formulations; rather, any medium suitable for incubation of placental ASC is encompassed. Any of the embodiments described herein for ASC and methods of their incubation and expansion may also be applied to CM generated via the described ASC/methods.

In certain embodiments, the described placental ASC have been cultured on a 2-dimensional (2D) substrate, a 3-dimensional (3D) substrate, or a combination thereof. Non-limiting examples of 2D and 3D culture conditions are provided in the Detailed Description and in the Examples.

Alternatively or in addition, the placental ASC are allogeneic to the subject; or, in other embodiments, are autologous; or, in other embodiments, are xenogeneic

Reference herein to “growth” of a population of cells is intended to be synonymous with expansion of a cell population. In certain embodiments, ASC (which may be, in certain embodiments, placental ASC), are expanded without substantial differentiation. In various embodiments, the described expansion is on a 2D substrate, on a 3D substrate, or a 2D substrate, followed by a 3D substrate.

Except where otherwise indicated, all ranges mentioned herein are inclusive.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to prepare the cells.

FIGS. 2A-J are plots of luminescence of Luminex® beads, reflective of concentration (vertical axis), for IL-1-ra, Collagen IV-1a, Fibronectin, IL-13, HGF, VEGF-A, IL-4, PDGF-AA, TIMP-1, TGFb2, and TGFb1 (in A-J, respectively) in conditioned medium batches. P250416 R21 and P150518 R02 are maternal batches; R090418 RO1 and R170216 R19 are fetal/serum batches; and PD060918 437B RO1 and PD08016 441 BR09 (also labeled as “PD08016 441B BR09”) are fetal SF batches. Bioreactor media from various batches (horizontal axis) were subjected to no treatment (BR; lanes 1-6 from left), Tangential Flow Filtration (TFF; Pall Corporation; lanes 7-12), or lyophilization (LYP; lanes 13-18) (upper panels). Lower panels depict analyses of CM generated in plates, with a higher cell/medium ratio.

FIG. 3 is a graph of secretion of IL-10 by PBMC in the absence or presence of ASC. Bars in each group, from left to right are: 1-3: Rat IL-10 after stimulation with 0, 1, or 10 mcg/ml LPS; and 4-6: human IL-10 after stimulation with 0, 1, or 10 mcg/ml LPS.

FIGS. 4A-B are charts depicting lymphocyte proliferation, measured by [³H]thymidine incorporation. Three replicates of each sample were performed. A. 2×10⁵ peripheral blood (PB)-derived MNC (donor A) were stimulated with an equal number of irradiated (3000 Rad) PB-derived MNCs (donor B) in an MLR test, in the presence of different amounts of ASC. B. PB-derived MNCs stimulated with ConA (1.5 mg/ml).

FIGS. 5A-C are charts depicting ASC regulation of pro- and anti-inflammatory cytokine secretion by human MNCs (isolated from peripheral blood). A-B depict secretion of IFN-gamma (A) and TNF-alpha (B) stimulation with ConA. C depicts secretion of IFN-gamma, TNF-alpha and IL-10 (left, middle, and right bars in each series, respectively) following stimulation with LPS. Supernatants were analyzed by ELISA

FIG. 6 is a graph of secretion profile of ASC under normoxic or hypoxic conditions.

FIGS. 7A-B are graphs (each split into 2 panels) depicting secretion, measured by fluorescence, of various factors following incubation of ASC with TNF-alpha+IFN-gamma (gray bars) or control media (black bars) in two separate experiments. C-D are graphs depicting fold-increase of secretion, measured by fluorescence, of GRO, IL-8, MCP-1, and RANTES (C), and IL-6, MCP-3, Angiogenin, Insulin-like Growth Factor Binding Protein-2 (IGFBP-2), Osteopontin, and Osteoprotegerin (D) following incubation of ASC with TNF-alpha alone, relative to incubation with control media (no cytokines).

FIGS. 8A-B are graphs depicting fold-increase relative to control medium (containing no cytokines) in secretion of MCP-1 (A) and GM-CSF (B) in several experiments, as measured by ELISA.

FIG. 9 is a plot showing RVSP (vertical axis) of nude mice given MCT and treated with (ordered from left to right) placebo or 15, 25, or 35×10⁶/kg. ASC.

FIGS. 10A-E are plots showing effect of ASC treatment on weight (A), exercise capacity (B), O₂ saturation (C), lung histology (D), and collagen deposition in the lungs as assessed by collagen (E) and hydroxyproline (F) content, following induction of lung fibrosis. For A-B, lanes from left to right are control; bleomycin (BL)+vehicle; and BL+batch #1 or batch #2 of ASC. For C, BL+vehicle was the group that dipped to 80%. For D, upper left, upper right, lower left, and lower right panels are control; BL+vehicle; and BL+batch #1 or batch #2 of ASC. For E-F, lanes from left to right are BL+vehicle; BL+batch #1 or batch #2 of ASC; and control,

FIG. 11A is a plot of day 21 necrosis as assessed PAS staining in untreated a low-dose ASC-treated mice, following induction of ischemia/reperfusion injury. Lanes from left to right are naive, IRI/untreated, IRI/IM-day 0, IRI/IM-day 1, IRI/IM-day 3, IRI/IV-day 0, IRI/IV-day 1, and IRI/IV-day 3. B-J are images of tissues stained for necrosis, for an untreated IRI mouse (B; 25.3% necrosis); low-dose IM ASC given on day 0 (C; 25.4% necrosis), 1 (D; 0.4% necrosis), or 3 (E; 2.3% necrosis); high-dose IM ASC given on day 1 (F; 0% necrosis) or 3 (G; 0% necrosis); and high-dose IV ASC given on day 0 (H; 4.5% necrosis), 1 (I; 4.5% necrosis), or 3 (J; 5.9% necrosis).

FIGS. 12A-B are plots of average (A) or individual (B) CRP levels (vertical axis) vs. days from ASC administration (horizontal axis). In B, data from individual subjects are represented by different symbols and/or line patterns. Levels after the first and second (where applicable) administration are shown as black and gray lines, respectively.

FIGS. 13A-B are plots of PEEP (Positive End Expiratory Pressure; A) and pH (B) (vertical axis) vs. days from ASC administration (horizontal axis).

FIGS. 14A-B are chest radiographs of a patient showing improvement after (B) vs. before (A) ASC administration.

FIG. 15 is a plot of creatinine of (vertical axis) vs. days from ASC administration (horizontal axis).

FIGS. 16A-J are plots of concentration of cytokines (vertical axis; units pg./mL.) of IFN-g (A-B), IL-2 (C-E), TNF-a (F-G), and CXCL-10 (H-J), in BALF (A, C, E, F, H, J) or serum (B, D, G, I). In E and J, both ASC-treated groups were combined into the single dataset. K-L are plots of lung injury score and alveolar thickening. Treatment groups are shown on horizontal axis. Asterisks above bars indicate significance vs. Naïve. P values of specific pairs are indicated: *p<0.05, **p<0.01, ***p<0.001, ****p<0.0001.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods and compositions that comprise placental adherent stromal cells (ASC) and their conditioned media (CM). In some embodiments, the ASC may be human ASC, or in other embodiments animal ASC.

In one embodiment, there is provided a method for treating, or in another embodiment reducing an incidence of, or in another embodiment ameliorating, a viral infection, comprising administering a composition that comprises a cultured placental ASC, thereby treating, reducing an incidence of, or ameliorating a viral infection. In another embodiment, there is provided a composition that comprises a cultured placental ASC, for treating, reducing an incidence of, or ameliorating, a viral infection. In certain embodiments, the cultured ASC has/have been incubated on a 3D substrate. In certain embodiments, the viral infection is a pulmonary infection. Pulmonary infection, as used herein, refers to an infection that comprises colonization of the lungs with the described virus; additional presence of the virus in other body tissues is not excluded. In other embodiments, there is provided a method of suppressing viral replication in a subject in need thereof, the method comprising the step of administering to the subject a composition comprising ASC. In still other embodiments, there is provided a method of reducing a viral load in a subject in need thereof, the method comprising the step of administering to the subject a composition comprising ASC.

In various embodiments, the placental ASC are maternal tissue-derived ASC (ASC from a maternal portion of the placenta); fetal tissue-derived ASC (ASC from a fetal portion of the placenta); or a mixture thereof. Alternatively or in addition, the placental ASC are allogeneic to the subject; or, in other embodiments, are autologous; or, in other embodiments, are xenogeneic. In certain embodiments, the composition is an injected composition, e.g., intramuscularly injected.

In another embodiment, there is provided a method for preventing, ameliorating, or reducing an incidence of deterioration of a subject with a viral infection, comprising administering a composition that comprises a cultured placental ASC, thereby preventing, ameliorating, or reducing an incidence of deterioration of a subject with a viral infection. In another embodiment, there is provided a composition that comprises a cultured placental ASC, for preventing, ameliorating, or reducing an incidence of deterioration of a subject with a viral infection. In certain embodiments, the viral infection is a pulmonary infection.

In another embodiment, there is provided a method for treating, or in another embodiment reducing an incidence of, or in another embodiment ameliorating, ARDS resulting from (or, in other embodiments, secondary to) a viral infection, comprising administering a composition that comprises a cultured placental ASC, thereby treating, reducing an incidence of, or ameliorating ARDS. In another embodiment, there is provided a composition that comprises a cultured placental ASC, for treating, reducing an incidence of, or ameliorating ARDS resulting from a viral infection. In certain embodiments, the ARDS is secondary to pneumonia, which may in turn be secondary to a viral infection. In other embodiments, the pneumonia is caused by a coronavirus, which is, in some embodiments, SARS-CoV-2. In some embodiments, the infection is a pulmonary infection.

Methods of identifying/diagnosing ARDS are known in the art. In certain embodiments, ARDS is characterized by bilateral infiltrates and hypoxemia, e.g., a decrease in the ratio of the partial pressure of arterial oxygen to the fraction of inspired oxygen (PaO₂/FiO₂). In further embodiments, the PF ratio is <300 mmHg, and the subject requires invasive mechanical ventilation, which may be, in certain embodiments, a minimum of 5 cm H₂0 PEEP (or Continuous positive airway pressure [CPAP]). In other embodiments, the subject has severe ARDS, e.g., a PF ratio of <100 mmHg In still embodiments, the subject has moderate ARDS, e.g., a PF ratio of 100-200 mmHg. In other embodiments, the subject has mild ARDS, e.g., a PF ratio of 200-300 mmHg As provided herein, ASC reduced PEEP (Positive End Expiratory Pressure) in patients with ARDS.

In yet another embodiment, there is provided a method for treating, or in another embodiment reducing an incidence of, or in another embodiment ameliorating, sepsis resulting from a viral infection, comprising administering a composition that comprises a cultured placental ASC, thereby treating, reducing an incidence of, or ameliorating sepsis. In certain embodiments, the cultured ASC has/have been incubated on a 3D substrate. In another embodiment, there is provided a composition that comprises a cultured placental ASC, for treating, reducing an incidence of, or ameliorating sepsis resulting from a viral infection. In certain embodiments, the sepsis is secondary to pneumonia, which may in turn be secondary to a viral infection. In other embodiments, the pneumonia is caused by a coronavirus, which is, in other embodiments, SARS-CoV-2. In specific embodiments, the sepsis is severe sepsis. In certain embodiments, the viral infection is a pulmonary infection.

In still another embodiment, there is provided a method for treating, or in another embodiment reducing an incidence of, or in another embodiment ameliorating, pulmonary hypertension resulting from a viral infection, comprising administering a composition that comprises a cultured placental ASC, thereby treating, reducing an incidence of, or ameliorating pulmonary hypertension. In another embodiment, there is provided a composition that comprises a cultured placental ASC, for treating, reducing an incidence of, or ameliorating pulmonary hypertension resulting from a viral infection. In certain embodiments, the pulmonary hypertension is secondary to pneumonia, which may in turn be secondary to a viral infection. In other embodiments, the pneumonia is caused by a coronavirus, which is, in other embodiments, SARS-CoV-2. In certain embodiments, the viral infection is a pulmonary infection.

In another embodiment, there is provided a method for treating, or in another embodiment reducing an incidence of, or in another embodiment ameliorating, multiple organ dysfunction syndrome resulting from a viral infection, comprising administering a composition that comprises a cultured placental ASC, thereby treating, reducing an incidence of, or ameliorating multiple organ dysfunction syndrome. In another embodiment, there is provided a composition that comprises a cultured placental ASC, for treating, reducing an incidence of, or ameliorating multiple organ dysfunction syndrome resulting from a viral infection. In certain embodiments, the multiple organ dysfunction syndrome is secondary to pneumonia, which may in turn be secondary to a viral infection. In other embodiments, the pneumonia is caused by a coronavirus, which is, in other embodiments, SARS-CoV-2. In certain embodiments, the viral infection is a pulmonary infection.

In further embodiments, there is provided a method for treating, or in another embodiment reducing an incidence of, or in another embodiment ameliorating, lung fibrosis resulting from a viral infection, comprising administering a composition that comprises a cultured placental ASC, thereby treating, reducing an incidence of, or ameliorating lung fibrosis. In another embodiment, there is provided a composition that comprises a cultured placental ASC, for treating, reducing an incidence of, or ameliorating lung fibrosis resulting from a viral infection. In certain embodiments, the cultured ASC has/have been incubated on a 3D substrate. In certain embodiments, the fibrosis is secondary to pneumonia, which may in turn be secondary to a viral infection. In other embodiments, the pneumonia is caused by a coronavirus, which is, in other embodiments, SARS-CoV-2. In certain embodiments, the viral infection is a pulmonary infection.

In yet other embodiments, there is provided a method for treating, or in another embodiment reducing an incidence of, or in another embodiment ameliorating, acute kidney injury (AKI), comprising administering a composition that comprises a cultured placental ASC, thereby treating, reducing an incidence of, or ameliorating AKI. In another embodiment, there is provided a composition that comprises a cultured placental ASC, for treating, reducing an incidence of, or ameliorating AKI resulting from a viral infection; e.g., a pulmonary infection. In certain embodiments, the AKI results from a viral infection. In other embodiments, the kidney injury is secondary to pneumonia and/or ARDS and/or accompanying sepsis—which may in turn be secondary to a viral infection. In other embodiments, the pneumonia is caused by a coronavirus, which is, in other embodiments, SARS-CoV-2. As provided herein, ASC reduce elevated creatinine levels in COVID-19 patients with ARDS.

In yet other embodiments, there is provided a method for treating, or in another embodiment reducing an incidence of, or in another embodiment ameliorating, AKI, comprising administering a composition that comprises a cultured ASC, wherein said ASC have been incubated on a 3D substrate, thereby treating, reducing an incidence of, or ameliorating AKI. In another embodiment, there is provided a composition that comprises a 3D-cultured ASC, for treating, reducing an incidence of, or ameliorating AKI resulting from a viral infection; e.g., a pulmonary infection. In certain embodiments, the AKI results from a viral infection. In other embodiments, the kidney injury is secondary to pneumonia and/or ARDS and/or accompanying sepsis—which may in turn be secondary to a viral infection. In other embodiments, the pneumonia is caused by a coronavirus, which is, in other embodiments, SARS-CoV-2. As provided herein, ASC reduce elevated creatinine levels in COVID-19 patients with ARDS.

In still other embodiments, there is provided a method for treating, or in another embodiment reducing an incidence of, or in another embodiment ameliorating, gastrointestinal injury resulting from a viral infection, comprising administering a composition that comprises a cultured placental ASC, thereby treating, reducing an incidence of, or ameliorating gastrointestinal injury. In another embodiment, there is provided a composition that comprises a cultured placental ASC, for treating, reducing an incidence of, or ameliorating gastrointestinal injury resulting from a viral infection. In certain embodiments, the pneumonia is caused by a coronavirus, which is, in other embodiments, SARS-CoV-2. In certain embodiments, the viral infection is a pulmonary infection.

In still other embodiments, there is provided a method for treating or ameliorating a coronavirus infection, comprising administering a composition that comprises a cultured placental ASC, thereby treating or ameliorating a coronavirus infection. In another embodiment, there is provided a composition that comprises a cultured placental ASC, for treating or ameliorating a coronavirus infection. In certain embodiments, the viral infection is a pulmonary infection.

In still other embodiments, there is provided a method for treating or ameliorating a coronavirus infection, comprising administering a composition that comprises a ASC, wherein said ASC have been incubated on a 3D substrate, thereby treating or ameliorating a coronavirus infection. In another embodiment, there is provided a composition that comprises a 3D-cultured ASC, for treating or ameliorating a coronavirus infection. In certain embodiments, the viral infection is a pulmonary infection.

In other embodiments, there is a provided a method of treating or reducing an incidence of a complication of a viral infection, e.g., a pulmonary infection, comprising administering a composition that comprises a cultured placental ASC. In certain embodiments, the described complication is at least one of vasculitis, lymphadenopathy, or both (a non-limiting example of which is Kawasaki disease). In other embodiments, the complication is pediatric multisystem inflammatory syndrome.

In other embodiments, there is a provided a method of treating or reducing an incidence of a complication of a viral infection, e.g., a pulmonary infection, comprising administering a composition that comprises a cultured ASC, wherein said ASC have been incubated on a 3D substrate. In certain embodiments, the described complication is at least one of vasculitis, lymphadenopathy, or both (a non-limiting example of which is Kawasaki disease). In other embodiments, the complication is pediatric multisystem inflammatory syndrome.

In still other embodiments, there is provided a method for treating, preventing, or ameliorating a pneumonia, comprising administering a composition that comprises a cultured placental ASC, wherein said pneumonia is associated with a viral infection, thereby treating, preventing, or ameliorating pneumonia. In certain embodiments, the viral infection is a pulmonary infection.

In still other embodiments, there is provided a method for treating, preventing, or ameliorating a complication of a pneumonia, comprising administering a composition that comprises a cultured placental ASC, wherein said pneumonia is associated with a viral infection, thereby treating, preventing, or ameliorating a complication of pneumonia. In certain embodiments, the viral infection is a pulmonary infection.

In still other embodiments, there is provided a method for treating, preventing, or ameliorating systemic inflammation, comprising administering a composition that comprises a cultured placental ASC, wherein said inflammation is associated with a viral infection, thereby treating, preventing, or ameliorating systemic inflammation. As provided herein, ASC sharply reduce CRP levels in COVID-19-infected subjects, particularly in subjects with strongly elevated CRP levels. In certain embodiments, the subject has a CRP level over 200 milligrams/milliliter (mg./mL.); or, in other embodiments, over 250 mg./mL., 300 mg./mL., or 350 mg./mL.; or, in yet other embodiments, between 200-400 mg./mL., 250-400 mg./mL., 300-400 mg./mL. or 350-400 mg./mL. In certain embodiments, the viral infection is a pulmonary infection.

In still other embodiments, there is provided a method for treating, preventing, or ameliorating a long-term sequela of a viral infection, comprising administering a composition that comprises a cultured placental ASC, thereby treating, preventing, or ameliorating a long-term sequela of a viral infection. In another embodiment, there is provided a composition that comprises a cultured placental ASC, for treating, reducing an incidence of, or ameliorating long-term sequela of a viral infection. In certain embodiments, the cultured ASC has/have been incubated on a 3D substrate. In certain embodiments, the virus is a coronavirus, which is, in other embodiments, SARS-CoV-2. Alternatively or in addition, the sequela is first observed after the patient tests negative (e.g., by a nasal swab) for an active viral infection; or, in other embodiments, at least 2, 3, 4, 5, 7, 10, 12, 18, or 24 weeks after the viral infection is believed to be resolved and/or testing negative. In certain embodiments, the sequela is fatigue. In other embodiments, the sequela is anxiety. In yet other embodiments, the sequela is shortness of breath. In still other embodiments, the sequela is sustained cough. In other embodiments, the sequela is limb pain (e.g., arm and/or leg pain), which may be, in various embodiments, a burning sensation, a tingling, or a feeling of unease in the affected limb(s), each of which represents a separate embodiment. In certain embodiments, the viral infection is a pulmonary infection.

In other embodiments, the coronavirus described herein is human and bat severe acute respiratory syndrome coronavirus (SARS-CoV) of the species Severe acute respiratory syndrome-related coronavirus, e.g. SARS-CoV-1 and SARS-CoV-2.

In other embodiments, the described virus is any virus in the family Coronaviridae, e.g. comprising Cornidovirineae (Orthocoronavirinae) and Letovirinae. In other embodiments, the described virus is any virus in the suborder Cornidovirineae, e.g., comprising Alphacoronaviruses, Betacoronaviruses, Gammacoronaviruses, and Deltacoronaviruses. In other embodiments, the described virus is any virus in the order Nidovirales, e.g., comprising Cornidovirineae, Tornidovirineae, Mesnidovirineae, Ronidovirineae, Nanidovirineae, and Arnidovirineae.

In other embodiments, the described virus is any virus in the realm Riboviria. Taxonomy of coronaviruses is known to those skilled in the art, and is described, e.g., in Siddell, S. G. et al. (Additional changes to taxonomy ratified in a special vote by the International Committee on Taxonomy of Viruses (October 2018). Arch. Virol. 164, 943-946 [2019]); Ziebuhr, J. et al. (Proposal 2017.013S. A.vl. Reorganization of the family Coronaviridae into two families, Coronaviridae (including the current subfamily Coronavirinae and the new subfamily Letovirinae) and the new family Tobaniviridae (accommodating the current subfamily Torovirinae and three other subfamilies), revision of the genus rank structure and introduction of a new subgenus rank. (ICTV, 2017); https://ictv.global/proposal/2017.Nidoovirales/.); Ziebuhr, J. et al. (Proposal 2019.021S.Ac.v1. Create ten new species and a new genus in the subfamily Orthocoronavirinae of the family Coronaviridae and five new species and a new genus in the subfamily Serpentovirinae of the family Tobaniviridae. (ICTV, 2019); and https://ictv.global/proposal/2019.Nidoovirales/.) Gorbalenya, A. E., Baker, S. C., Baric, R. S. et al. The species Severe acute respiratory syndrome-related coronavirus: classifying 2019-nCoV and naming it SARS-CoV-2. Nat Microbiol (2020).

In certain embodiments, the treated virus is SARS-CoV-2, e.g., a virus having a sequence at least 96%, or in other embodiments, at least 91%, at least 92%, at least 93%, at least 94%, at least 95%, at least 96%, at least 97%, at least 98%, or at least 99% homologous to at least 1 sequence selected from the nucleotide sequences set forth in GenBank Accession Nos. NC_045512.2, MT126808, MT123290, MT093571, MT066176, MT263074, MT276331, MT233523, MT066156, and LC528233 (SEQ ID NOs. 1-10).

In other embodiments, the treated coronavirus has a sequence at least 96%, or in other embodiments, at least 91%, at least 92%, at least 93%, at least 94%, at least 95%, at least 96%, at least 97%, at least 98%, or at least 99% homologous to at least 1 sequence selected from the nucleotide sequences set forth in GenBank Accession Nos. NC_004718.3, AY274119.3, GU553363.1, DQ182595.1, AY297028.1, and AY515512.1 (SEQ ID NOs. 11-16).

In other embodiments, the treated virus is related to a bat coronavirus, e.g., related to GenBank Accession No. DQ022305 (SEQ ID NO. 17).

In another embodiment, there is provided use of ASC for the manufacture of a medicament for treating or ameliorating any of the diseases, disorders, and complications mentioned herein, each of which represents a separate embodiment.

In still other embodiments, there is provided an article of manufacture, comprising (a) a packaging material, wherein the packaging material comprises a label for use in any of the diseases, disorders, and complications mentioned herein, each of which represents a separate embodiment; and (b) a pharmaceutical composition comprising ASC. In some embodiments, the pharmaceutical composition is frozen. In other embodiments, the label indicates use in suppressing viral replication. In still other embodiments, the label indicates use in reducing viral load. As provided herein, administration of a therapeutically effective amount of ASC is useful in treating viral infections.

In other embodiments, there is provided a method of treating hypercytokinemia (also known, in some embodiments, as “cytokine storm”), the method comprising the step of administering to the subject a therapeutically effective amount of ASC, thereby treating the hypercytokinemia in the subject. Methods of diagnosing and tracking the progression of hypercytokinemia are well known in the art, and include, inter alia, measurement of serum cytokine levels and/or expression in PBMC of cytokines, chemokines, and/or death proteins such as TRAIL, as described in Bray M et al., 2001, Yen J Y et al., 2011, and the references cited therein.

In other embodiments, there is provided a method of treating systemic inflammatory response syndrome (SIRS), the method comprising the step of administering to the subject a therapeutically effective amount of ASC, thereby treating the SIRS in the subject. Methods of diagnosing and tracking the progression of SIRS are well known in the art, and include, inter alia, measurement of serum cytokine levels and/or expression in PBMC of cytokines, chemokines, death proteins such as TRAIL, and/or fibrin-related genes as described in Bray M et al., 2001, Geisbert T W et al., 2003, Yen J Y et al., 2011, and the references cited therein.

In another embodiment is provided use of ASC for the manufacture of a medicament identified for treating hypercytokinemia or SIRS. In still other embodiments, there is provided a pharmaceutical composition for treating hypercytokinemia or SIRS, comprising the described ASC.

In certain embodiments, the herein-described SIRS patient exhibits leukopenia, e.g., an absolute WBC count below 3500 cells per microliter [/μL]); or, in other embodiments, below 3000, 2500, 2000, 1500, or 1000 cells/μL, e.g., for an adult. Alternatively, the subject exhibits leukocytosis, e.g., an absolute WBC count above >10,000 cells/μL; or in other embodiments, above 10,500, 11,000, 12,000, 13,000, 15,000, 18,000, or 20,000 cells/μL, e.g., for an adult.

Alternatively or in addition, the SIRS patient exhibits both leukopenia and lymphopenia (83.2%), e.g., a lymphocyte count below 1000/μL; or, in other embodiments, below 800, 600, 500, 400, 300, 200, or 100 cells/μL, e.g., for an adult; and/or (in different embodiments) thrombocytopenia, e.g., a platelet count below 50,000/μL; or, in other embodiments, below 100,000, 80,000, 60,000, 40,000, 30,000, 20,000, or 20,000/μL, e.g., for an adult.

In still other embodiments, the SIRS patient exhibits elevated levels of C-reactive protein; e.g., greater than 3 milligrams per liter (mg/L); or, in other embodiments, greater than 4, 5, 6, 8, or 10 mg/L. Each embodiment of SIRS symptoms and criteria may be freely combined, except where self-contradictory (e.g., leukopenia and leukocytosis).

In other embodiments, there is provided a method of treating dysregulated coagulation, the method comprising the step of administering to the subject a therapeutically effective amount of ASC, thereby treating the dysregulated coagulation in the subject. Methods of diagnosing and tracking the progression of dysregulated coagulation are well known in the art, and include, inter alia, measurement of intravascular coagulation, e.g. using ROTEM® delta, available from Tem International GmbH, or as described in Monaca E et al., 2014; and expression in PBMC of fibrin-related genes, as described in Bray M et al., 2001, Geisbert T W et al., 2003, Yen J Y et al., 2011, and the references cited therein.

In another embodiment is provided use of ASC for the manufacture of a medicament identified for treating dysregulated coagulation. In still other embodiments, there is provided a pharmaceutical composition for treating dysregulated coagulation, comprising the described ASC.

In other embodiments, there is provided a method of treating septic shock, the method comprising the step of administering to the subject a therapeutically effective amount of ASC, thereby treating septic shock in the subject. Methods of diagnosing and tracking the progression of septic shock are well known in the art, and include, inter alia, measurement of expression levels of tissue factor in primate monocytes and/or macrophages, as described in Bray M et al., 2003, and the references cited therein. In some embodiments, the septic shock is secondary to a hemorrhagic fever virus. In other cases, septic shock may be caused by other pathogens, for example influenza virus. In still other cases, septic shock may be independent of a pathogen, or in other embodiments may be of unknown etiology. In another embodiment is provided use of ASC for the manufacture of a medicament identified for treating septic shock. In still other embodiments, there is provided a pharmaceutical composition for treating septic shock, comprising the described ASC.

In various embodiments, the described placental ASC are maternal tissue-derived ASC; fetal tissue-derived ASC; or a mixture thereof. Alternatively or in addition, the placental ASC are allogeneic to the subject; or, in other embodiments, are autologous; or, in other embodiments, are xenogeneic. In other embodiments, conditioned medium (CM) of placental ASC is utilized in place of ASC. In other embodiments, the composition is an injected composition, e.g., intramuscularly injected. Any of these embodiments may be freely combined with any of the therapeutic embodiments mentioned herein.

As provided herein, placental ASC and CM exhibit ability to ameliorate inflammation, ischemia/reperfusion injury, muscle trauma, irradiation and hematological disorders; to modulate and support recovery of various organ systems, e.g., by inducing regeneration and modulating undesired inflammation; and in treating pulmonary hypertension, lung fibrosis, acute kidney injury, and gastrointestinal injury.

In still other embodiments, there is provided a composition for treating or ameliorating any of the diseases, disorders, and complications mentioned herein, each of which represents a separate embodiment, comprising cultured placental ASC. In other embodiments, the composition comprises placental ASC conditioned medium (“ASC-CM”).

In yet other embodiments, the described placenta-derived ASC secrete elevated levels of RANTES (C-C motif chemokine 5; UniProt No. P13501). In some embodiments, the RANTES secretion is measured after removing the cells from the bioreactor. In certain embodiments, the RANTES secretion is at least 2-fold, 3-fold, 4-fold, 5-fold, 6-fold, 8-fold, 10-fold, 15-fold, 20-fold, 30-fold, 50-fold, 70-fold, 100-fold, 150-fold, 200-fold, 300-fold, 500-fold, 700-fold, or 1000-fold as high as cells prepared in the absence of added cytokines. In certain embodiments, as provided herein, RANTES secretion is measured by incubating 5×10⁵ ASC for 24 hours under standard conditions, then replacing the medium with serum medium and incubating for an additional 24 hours. In more specific embodiments, at least 20 picrograms (pg.), 30 pg., 50 pg., 70 pg., 100 pg., 150 pg., 200 pg., 300 pg., 400 pg., 500 pg., 700 pg., 1000 pg., 1500 pg., 2000 pg., 3000 pg., 4000 pg., 5000 pg., 6000 pg., 8000 pg., 10,000 pg., 15,000 pg., or 20,000 pg. of RANTES are secreted by the cells under these conditions. In other embodiments, there is provided a use of these ASC in any of the therapeutic embodiments described herein, each of which represents a separate embodiment.

### Basal Media for Expansion of ASC

Those skilled in the art will appreciate that growth media are utilized to expand the described placental ASC and/or produce the described CM for the compositions and methods described herein. Non-limiting examples of base media useful in 2D and 3D culturing include Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's sale base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153, and mixtures thereof in any proportions. In certain embodiments, DMEM is used. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be FBS, or in other embodiments another animal serum.

Alternatively or in addition, the medium may be supplemented by growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

The various media described herein, i.e., the 2D growth medium and the 3D growth medium, may be independently selected from each of the described embodiments relating to medium composition. In various embodiments, any medium suitable for growth of cells in a standard tissue apparatus and/or a bioreactor may be used.

It will also be appreciated that in certain embodiments, when the described ASC are intended for administration to a human subject, the cells and the culture medium (e.g., with the above-described medium additives) are substantially xeno-free, i.e., devoid of any animal contaminants e.g., mycoplasma. For example, the culture medium can be supplemented with a serum-replacement, human serum and/or synthetic or recombinantly produced factors.

### ASC and Sources Thereof

With reference to placenta-derived ASC, except where indicated otherwise, “placenta”, “placental tissue”, and the like, as used herein, refer to any portion of the placenta. Placenta-derived ASC may be obtained, in various embodiments, from either fetal or, in other embodiments, maternal regions of the placenta, or in other embodiments, from both regions. More specific embodiments of maternal sources are decidua regions (e.g., decidua basalis, decidua capsularis, and decidua parietalis). More specific embodiments of fetal sources are amnion and chorion, including villous chorion. In certain embodiments, tissue specimens are washed in a physiological buffer, non-limiting examples of which are phosphate-buffered saline (PBS) and Hank's buffer. In certain embodiments, the placental tissue from which ASC are harvested includes at least one of the chorionic and decidua regions of the placenta, or, in still other embodiments, both the chorionic and decidua regions of the placenta. More specific embodiments of chorionic regions are chorionic mesenchymal and chorionic trophoblastic tissue. In a non-limiting embodiment, a mixture of maternal and fetal placental cells is obtained by mincing whole placenta; or, in other embodiments, a portion thereof; or, in still other embodiments, whole placenta, apart from the amnion, chorion, and umbilical cord.

Placental cells may be obtained, in various embodiments, from a full-term or pre-term placenta. In some embodiments, the placental tissue is optionally minced, followed by enzymatic digestion. Single-cell suspensions can be made, in other embodiments, by treating the tissue with a digestive enzyme (see below) or/and physical disruption, a non-limiting example of which is mincing and flushing the tissue parts through a nylon filter or by gentle pipetting (e.g., Falcon, Becton, Dickinson, San Jose, CA) with washing medium. In some embodiments, the tissue treatment includes use of a DNAse, a non-limiting example of which is Benzonase from Merck.

Optionally, residual blood is removed from the placenta before cell harvest. This may be done by a variety of methods known to those skilled in the art, for example by perfusion. “Perfuse” or “perfusion” herein refers to pouring or passaging a fluid over or through an organ or tissue. In certain embodiments, the placental tissue may be from any mammal, while in other embodiments, the placental tissue is human A convenient source of placental tissue is a post-partum placenta (e.g., less than 10 hours after birth), however, a variety of sources of placental tissue or cells may be contemplated by the skilled person. In other embodiments, the placenta is used within 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to an adherent material (e.g., configured as a surface) to thereby isolate adherent cells. In some embodiments, the donor is 35 years old or younger, while in other embodiments, the donor may be any woman of childbearing age.

Placenta-derived cells can be propagated, in some embodiments, by using a combination of 2D and 3D culturing conditions. Conditions for propagating adherent cells in 2D and 3D culture are further described hereinbelow and in the Examples section which follows.

Those skilled in the art will appreciate, in light of the present disclosure, that cells may be, in some embodiments, extracted from a placenta, for example using physical and/or enzymatic tissue disruption, followed by marker-based cell sorting, and then may be subjected to the culturing methods described herein.

### Treatment of Cells With Pro-Inflammatory Cytokines

In certain embodiments of the described methods and compositions, the composition of the medium is not varied during the course of the culturing process used to expand the placental ASC that are used in the described methods and compositions and/or for producing the described CM. In other words, no attempt is made to intentionally vary the medium composition by adding or removing factors or adding fresh medium with a different composition than the previous medium. Reference to varying the composition of the medium does not include variations in medium composition that automatically occur as a result of prolonged culturing, for example due to the absorption of nutrients and the secretion of metabolites by the cells therein, as will be appreciated by those skilled in the art.

In other embodiments, the method used to expand the steps comprises 2D culturing, followed by 3D culturing. In certain embodiments, the 3D culturing method comprises the sub-steps of: (a) incubating ASC in a 3D culture apparatus in a first growth medium, wherein no inflammatory cytokines have been added to the first growth medium; and (b) subsequently incubating the ASC in a 3D culture apparatus in a second growth medium, wherein one or more pro-inflammatory cytokines have been added to the second growth medium. Those skilled in the art will appreciate, in light of the present disclosure, that the same 3D culture apparatus may be used for the incubations in the first and second growth medium by simply adding cytokines to the medium in the culture apparatus, or, in other embodiments, by removing the medium from the culture apparatus and replacing it with medium that contains cytokines. In other embodiments, a different 3D culture apparatus may be used for the incubation in the presence of cytokines, for example by moving (e.g. passaging) the cells to a different incubator, before adding the cytokine-containing medium.

Other embodiments of pro-inflammatory cytokines, and methods comprising same, are described in WO 2017/141181 to Pluristem Ltd, by Zami Aberman et al., which is incorporated by reference herein.

### Serum-Free and Serum Replacement Media

In other embodiments, the described cell populations are produced by expanding a population of placental ASC in a medium that contains less than 5% animal serum. In certain embodiments, the cell population contains at least predominantly fetal cells (referred to as a “fetal cell population”), or, in other embodiments, contains at least predominantly maternal cells (a “maternal cell population”). In certain embodiments, the aforementioned medium contains less than 4%; less than 3%; less than 2%; less than 1%; less than 0.5%; less than 0.3%; less than 0.2%; or less than 0.1% animal serum. In other embodiments, the medium does not contain animal serum. In other embodiments, the medium is a defined medium to which no serum has been added. Low-serum and serum-free media are collectively referred to as “serum-deficient medium/media”.

Those skilled in the art will appreciate that reference herein to animal serum includes serum from any species, provided that the serum stimulates expansion of the ASC population, for example human serum, bovine serum (e.g. fetal bovine serum and calf bovine serum), equine serum, goat serum, and porcine serum.

In other embodiments, the described cell populations are produced by a process comprising: a. incubating the ASC population in a first medium, wherein the first medium contains less than 5% animal serum, thereby obtaining a first expanded cell population; and b. incubating the first expanded cell population in a second medium, wherein the second medium also contains less than 5% animal serum, and wherein one or more activating components are added to the second medium. This second medium can also be referred to herein as an activating medium. In other embodiments, the first medium or the second medium, or in other embodiments both the first and second medium, is/are serum free. In still other embodiments, the first medium contains a first basal medium, with the addition of one or more growth factors, collective referred to as the “first expansion medium” (to which a small concentration of animal serum is optionally added); and the activating medium contains a second basal medium with the addition of one or more growth factors (the “second expansion medium”), to which activating component(s) are added. In more specific embodiments, the second expansion medium is identical to the first expansion medium; while in other embodiments, the second expansion medium differs from the first expansion medium in one or more components.

In certain embodiments, the aforementioned step of incubating the ASC population in a first medium is performed for at least 17 doublings, or in other embodiments at least 6, 8, 12, 15, or at least 18 doublings; or 12-30, 12-25, 15-30, 15-25, 16-25, 17-25, or 18-25 doublings.

In other embodiments, the ASC population is incubated in the second medium for a defined number of days, for example 4-10, 5-10, 6-10, 4-9, 4-8, 4-7, 5-9, 5-8, 5-7, 6-10, 6-9, or 6-8; or a defined number of population doublings, for example at least 3, at least 4, at least 5, at least 6, 3-10, 3-9, 3-8, 4-10, 4-9, or 4-8. The cells are then subjected to additional culturing in the second medium in a bioreactor. In some embodiments, the bioreactor culturing is performed for at least 4, at least 5, at least 6, at least 7, at least 8, at least 9, 4-10, 4-9, 4-8, 5-10, 5-9, 5-8, 6-10, 6-9, or 6-8 population doublings; or, in other embodiments, for at least 4, at least 5, at least 6, at least 7, 4-15, 4-12, 4-10, 4-9, 4-8, 4-7, 4-15, 5-12, 5-10, 5-9, 5-8, 5-7, 6-15, 6-12, 6-10, 6-9, 6-8, or 6-7 days. In certain embodiments, the bioreactor contains 3D carriers, on which the cells are cultured.

In still other embodiments, ASC are extracted from placenta into serum-containing medium. A non-limiting extraction protocol is described in Example 1 of International Patent Application WO 2016/098061, in the name of Esther Lukasiewicz Hagai et al., published on Jun. 23, 2016, which is incorporated herein by reference in its entirety. Following initial extractions, cells are, in further embodiments, expanded in SRM. For such embodiments, the nomenclature of the aforementioned steps is retained, with the first medium (serum-replacement medium or SRM) called the “first medium”, and the activating medium called the “second [or activating] medium”.

In certain embodiments, the described serum-deficient medium is supplemented with factors intended to stimulate cell expansion in the absence of serum. Such medium is referred to herein as serum-replacement medium or SRM, and its use, for example in cell culture and expansion, is known in the art, and is described, for example, in Kinzebach et al. In still other embodiments, a chemically-defined medium is utilized.

In certain embodiments, the described SRM comprises bFGF (basic fibroblast growth factor, also referred to as FGF-2), TGF-β (TGF-β, including all isotypes, for example TGFβ1, TGFβ2, and TGFβ3), or a combination thereof. In other embodiments, the SRM comprises bFGF, TGF-β, and PDGF. In still other embodiments, the SRM comprises bFGF and TGF-β, and lacks PDGF-BB. Alternatively or in addition, insulin is also present. In still other embodiments, an additional component selected from ascorbic acid, hydrocortisone and fetuin is present; 2 components selected from ascorbic acid, hydrocortisone and fetuin are present; or ascorbic acid, hydrocortisone and fetuin are all present.

Other SFM and SRM embodiments are disclosed in international patent application publ. no. WO 2019/186471, filed on Mar. 28, 2019, in the name of Lior Raviv et al., which is incorporated herein by reference.

### Other Embodiments of Placenta-Derived ASC

In certain embodiments, the described ASC are plastic adherent under standard culture conditions, express the surface molecules CD105, CD73 and CD90, and do not express CD45, CD34, CD14 or CD11 b, CD79α, CD19 and HLA-DR. As used herein, the phrase plastic adherent refers to cells that are capable of attaching to a plastic attachment substrate and expanding or proliferating on the substrate. In some embodiments, the cells are anchorage dependent, i.e., require attachment to a surface in order to proliferate grow in vitro.

In still other embodiments, the described placenta-derived ASC (which hereinafter refers to the cells used in the described methods and compositions, or, in other embodiments, cells used to produce CM, that are used in the described methods and compositions) are a mixture of fetal-derived placental ASC (also referred to herein as “fetal ASC” or “fetal cells”) and maternal-derived placental ASC (also referred to herein as “maternal ASC” or “maternal cells”) and contains predominantly maternal cells. In more specific embodiments, the mixture contains at least 80%, at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% maternal cells; or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96-99.5%, 97-99.5%, 98-99.5%, 90-99.9%, 91-99.9%, 92-99.9%, 93-99.9%, 94-99.9%, 95-99.9%, 96-99.9%, 97-99.9%, 98-99.9% % maternal cells.

In yet other embodiments, the described cells are predominantly or completely maternal cell preparations, or are predominantly or completely fetal cell preparations, each of which represents a separate embodiment. Predominantly or completely maternal cell preparations may be obtained by methods known to those skilled in the art, including the protocol detailed in Example 1 and the protocols detailed in PCT Publ. Nos. WO 2007/108003, WO 2009/037690, WO 2009/144720, WO 2010/026575, WO 2011/064669, and WO 2011/132087. The contents of each of these publications are incorporated herein by reference. Predominantly or completely fetal cell preparations may be obtained by methods known to those skilled in the art, including selecting fetal cells via their markers (e.g., a Y chromosome in the case of a male fetus), and expanding the cells. In certain embodiments, maternal cell populations are used in the described methods and compositions. In other embodiments, fetal cells are used.

In other embodiments, the described cells are a population that does not contain a detectable amount of maternal cells and is thus entirely fetal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

In still other embodiments, the preparation is a mixture of fetal and maternal cells and is enriched for fetal cells. In more specific embodiments, the mixture contains at least 70% fetal cells. In more specific embodiments, at least about 30%, 35%, 40%, 45%, 50%, 55%, 60%, 65%, 75%, 80%, 85%, 90%, 95%, 96%, 97%, 98%, or 99% of the cells are fetal cells. Expression of CD200, as measured by flow cytometry, using an isotype control to define negative expression, can be used as a marker of fetal cells under some conditions. In yet other embodiments, at least 60%, 65%, 70%, 75%, 80%, 85%, 90%, 92%, 95%, 96%, 97%, 98%, 99%, or 99.5% of the described cells are fetal cells.

In more specific embodiments, the mixture contains 20-80% fetal cells; 30-80% fetal cells; 40-80% fetal cells; 50-80% fetal cells; 60-80% fetal cells; 20-90% fetal cells; 30-90% fetal cells; 40-90% fetal cells; 50-90% fetal cells; 60-90% fetal cells; 20-80% maternal cells; 30-80% maternal cells; 40-80% maternal cells; 50-80% maternal cells; 60-80% maternal cells; 20-90% maternal cells; 30-90% maternal cells; 40-90% maternal cells; 50-90% maternal cells; or 60-90% maternal cells.

In certain embodiments, the described ASC are distinguishable from human mesenchymal stromal cells (MSC)—which may, e.g., be isolated from bone marrow—as defined by The Mesenchymal and Tissue Stem Cell Committee of the ISCT (Dominici et al., 2006), based on the following 3 criteria: 1. Plastic-adherence when maintained in standard culture conditions (a minimal essential medium +20% fetal bovine serum (FBS)). 2. Expression of the surface molecules CD105, CD73 and CD90, and lack of expression of CD45, CD34, CD14 or CD11b, CD79α or CD19 and HLA-DR. 3. Ability to differentiate into osteoblasts, adipocytes and chondroblasts in vitro. By contrast, the described placental ASC are, in certain embodiments, characterized by a reduced differentiation potential, as exemplified and described further herein.

### Surface Markers and Additional Characteristics of ASC

Alternatively or additionally, the described ASC (which are used in the described methods and compositions, or to produce CM) may express a marker or a collection of markers (e.g. surface marker) characteristic of MSC or mesenchymal-like stromal cells. In some embodiments, the ASC express some or all of the following markers: CD105 (UniProtKB Accession No. P17813), CD29 (Accession No. P05556), CD44 (Accession No. P16070), CD73 (Accession No. P21589), and CD90 (Accession No. P04216). In some embodiments, the ASC do not express some or all of the following markers: CD3 (e.g. Accession Nos. P09693 [gamma chain] P04234 [delta chain], P07766 [epsilon chain], and P20963 [zeta chain]), CD4 (Accession No. P01730), CD11b (Accession No. P11215), CD14 (Accession No. P08571), CD19 (Accession No. P15391), and/or CD34 (Accession No. P28906). In more specific embodiments, the ASC also lack expression of CD5 (Accession No. P06127), CD20 (Accession No. P11836), CD45 (Accession No. P08575), CD79-alpha (Accession No. B5QTD1), CD80 (Accession No. P33681), and/or HLA-DR (e.g. Accession Nos. P04233 [gamma chain], P01903 [alpha chain], and P01911 [beta chain]). The aforementioned, non-limiting marker expression patterns were found in certain maternal placental cell populations that were expanded on 3D substrates. All UniProtKB entries mentioned in this paragraph were accessed on Jul. 7, 2014. Those skilled in the art will appreciate that the presence of complex antigens such as CD3 and HLA-DR may be detected by antibodies recognizing any of their component parts, such as, but not limited to, those described herein.

In some embodiments, the ASC possess a marker phenotype that is distinct from bone marrow-mesenchymal stem cells (BM-MSC). In certain embodiments, the ASC are positive for expression of CD10 (which occurs, in some embodiments, in both maternal and fetal ASC); are positive for expression of CD49d (which occurs, in some embodiments, at least in maternal ASC); are positive for expression of CD54 (which occurs, in some embodiments, in both maternal and fetal ASC); are bimodal, or in other embodiments positive, for expression of CD56 (which occurs, in some embodiments, in maternal ASC); and/or are negative for expression of CD106. Except where indicated otherwise, bimodal refers to a situation where a significant percentage (e.g., at least 20%) of a population of cells express a marker of interest, and a significant percentage do not express the marker.

“Positive” expression of a marker indicates a value higher than the range of the main peak of an isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. “High” expression of a marker, and term “highly express[es]” indicates an expression level that is more than 2 standard deviations higher than the expression peak of an isotype control histogram, or a bell-shaped curve matched to said isotype control histogram.

A cell is said to express a protein or factor if the presence of protein or factor is detectable by standard methods, an example of which is a detectable signal using fluorescence-activated cell sorting (FACS), relative to an isotype control. Reference herein to “secrete”/“secreting”/“secretion” relates to a detectable secretion of the indicated factor, above background levels in standard assays. For example, 0.5×10⁶ fetal or maternal ASC can be suspended in 4 ml medium (DMEM+10% FBS+2 mM L-Glutamine), added to each well of a 6 well-plate, and cultured for 24 hrs. in a humidified incubator (5% CO₂, at 37° C.). After 24 h, DMEM is removed, and cells are cultured for an additional 24 hrs in 1 ml RPMI 1640 medium+2 mM L-Glutamine+0.5% HSA. The CM is collected from the plate, and cell debris is removed by centrifugation.

According to some embodiments, the described ASC are capable of suppressing an immune reaction in the subject. Methods of determining the immunosuppressive capability of a cell population are well known to those skilled in the art, with exemplary methods described in Example 3 of PCT Publication No. WO 2009/144720, which is incorporated herein by reference in its entirety. For example, in an exemplary, non-limiting mixed lymphocyte reaction (MLR) assay, irradiated cord blood (iCB) cells, for example human cells or cells from another species, are incubated with peripheral blood-derived monocytes (PBMC; e.g., human PBMC or PBMC from another species), in the presence or absence of a cell population to be tested. PBMC cell replication, which correlates with the intensity of the immune response, can be measured by a variety of methods known in the art, for example by ³H-thymidine uptake. Reduction of the PBMC cell replication when co-incubated with test cells indicates an immunosuppressive capability. Alternatively, a similar assay can be performed with peripheral blood (PB)-derived MNC, in place of CB cells. Alternatively or in addition, secretion of pro-inflammatory and anti-inflammatory cytokines by blood cell populations (such as CB cells or PBMC) can be measured when stimulated (for example by incubation with non-matched cells, or with a non-specific stimulant such as PHA), in the presence or absence of the ASC. In certain embodiments, for example in the case of human ASC, as provided in WO 2009/144720, when 150,000 ASC are co-incubated for 48 hours with 50,000 allogeneic PBMC, followed by a 5-hour stimulation with 1.5 mcg/ml of LPS, the amount of IL-10 secretion by the PBMC is at least 120%, at least 130%, at least 150%, at least 170%, at least 200%, or at least 300% of the amount observed with LPS stimulation in the absence of ASC.

In still other embodiments, the ASC secrete immunoregulatory factor(s). In certain embodiments, the ASC secrete a factor selected from TNF-beta (UniProt identifier P01374) and Leukemia inhibitory factor (LIF; UniProt identifier P15018). In other embodiments, ASC secrete a factor selected from MCP-1 (CCL2), Osteoprotegerin, MIF (Macrophage migration inhibitory factor; Accession No. P14174), GDF-15, SDF-1 alpha, GROa (Growth-regulated alpha protein; Accession No. P09341), beta2-Microglobulin, IL-6, IL-8 (UniProt identifier P10145), TNF-beta, ENA78/CXCL5, eotaxin/CCL11 (Accession No. P51671), and MCP-3 (CCL7). In certain embodiments, the ASC secrete MCP-1, Osteoprotegerin, MIF, GDF-15, SDF-1 alpha, GROa, beta2-Microglobulin, IL-6, IL-8, TNF-beta, and MCP-3, which were found to be secreted by maternal cells. In other embodiments, the ASC secrete MCP-1, Osteoprotegerin, MIF, GDF-15, SDF-1 alpha, beta2-Microglobulin, IL-6, IL-8, ENA78, eotaxin, and MCP-3, which were found to be secreted by fetal cells. UniProt entries in this paragraph were accessed on Mar. 23, 2017.

In yet other embodiments, the ASC secrete anti-fibrotic factor(s). In certain embodiments, the ASC secrete a factor selected from Serpin E1 (Plasminogen activator inhibitor 1; Uniprot Accession No. P05121) and uPAR (Urokinase plasminogen activator surface receptor; Uniprot Accession No. Q03405). In other embodiments, the ASC secrete factors that facilitate. In still other embodiments, the ASC secrete Serpin E1 and uPAR, which were found to be secreted by maternal and fetal cells. All UniProt entries in this paragraph were accessed on Apr. 3, 2017.

In other embodiments, the ASC secrete a factor(s) that promotes extracellular matrix (ECM) remodeling. In certain embodiments, the ASC secrete a factor selected from TIMP1, TIMP2, MMP-1, MMP-2, and MMP-10. In other embodiments, the ASC secrete TIMP1, TIMP2, MMP-1, MMP-2, and MMP-10, which were found to be secreted by maternal cells. In still other embodiments, the ASC secrete TIMP1, TIMP2, MMP-1, and MMP-10, which were found to be secreted by fetal cells.

In general, in certain embodiments, the described ASC exhibit a spindle shape when cultured under 2D conditions, or more specifically, are spindle in shape, with a flat, polygonal morphology, and are 15-19 μM in diameter. Alternatively or in addition, at least 90% of the cells are Oct-4 minus, as assessed by FACS. In certain embodiments, further steps of purification or enrichment for ASC may be performed. Such methods include, but are not limited to, FACS using ASC marker expression. In other embodiments, the described cells have not been subject to any type of cell sorting in the process used to isolate them. Cell sorting, in this context, refers to any a procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

Alternatively or in addition, the ASC (a) have a Population Doubling Level (PDL) of no more than 25; (b) stimulate endothelial cell proliferation and/or bone marrow migration in in vitro assays (for example, as described herein); (c) secrete, in various embodiments, at least 1, at least 2, at least 3, at least 4, at least 5, at least 6, or all 7 of IL-10, VEGF, Angiogenin, Osteopontin, IL-6, IL-8, MCP-1; (d) exhibit normal karyotype; (e) exhibit expression (in various embodiments, in at least 80%, 85%, 90%, 93%, 95%, 97%, or 98% of the cells) of CD105, CD73, CD29, and CD90; (f) exhibit lack of expression (in various embodiments, in at least 90%, 93%, 95%, 97%, 98%, 99%, 99.5%, 99.7%, or 99.75% of the cells) of CD14, CD19, CD31, CD34, CD45, HLA-DR, and CD235; or any combination of 2 or more of characteristics a-f, each of which represents a separate embodiment. Alternatively or in addition, less than 5%, less than 10%, less than 20%, less than 30%, less than 40%, less than 50%, more than 60%, more than 70%, more than 80%, more than 90%, or more than 95% of the cells express CD200. These possibilities may be independently combined with characteristics a-f and combinations thereof, each of which represents a separate embodiment.

In other embodiments, each of CD29, CD73, CD90, and CD105 is expressed by more than 80% of the ASC in each of the populations; and over 90% (or in other embodiments, over 95%, or over 98%) of the cells in each population are resistant to osteogenesis, as described in WO 2016/098061, which is incorporated herein by reference. In some embodiments, differentiation into osteocytes is assessed by incubation for 17 days with a solution containing 0.1 mcM dexamethasone, 0.2 mM ascorbic acid, and 10 mM glycerol-2-phosphate, in plates coated with vitronectin and collagen (standard osteogenesis induction conditions). In yet other embodiments, each of CD34, CD39, and CD106 is expressed by less than 10% of the cells; less than 20% of the cells highly express CD56; and the cells do not differentiate into osteocytes, after incubation under the standard conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56, and/or the cells do not differentiate into osteocytes, after incubation under the standard conditions. In still other embodiments, the conditions are incubation for 26 days with a solution containing 10 mcM dexamethasone, 0.2 mM ascorbic acid, 10 mM glycerol-2-phosphate, and 10 nM Vitamin D, in plates coated with vitronectin and collagen (modified osteogenesis induction conditions). The aforementioned solutions will typically contain cell culture medium such as DMEM+10% serum or the like, as will be appreciated by those skilled in the art. In yet other embodiments, less than 20%, 15%, or 10% of the described cells highly express CD56. In various embodiments, the cell population may be less than 50%, 40%, 30%, 20%, 10%, or 5% positive for CD200. In other embodiments, the cell population is more than 50%, 60%, 70%, 80%, 90%, 95%, 97%, 98%, 99%, or 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetal tissue-derived placental cell populations that were expanded on 3D substrates, as provided herein.

In other embodiments, each of CD29, CD73, CD90, and CD105 is expressed by more than 80% of each of the ASC populations; and over 90% (or in other embodiments, over 95%, or over 98%) of the cells in each population are resistant to adipogenesis, as described in WO 2016/098061, which is incorporated herein by reference. In some embodiments, differentiation into adipocytes is assessed by incubation in adipogenesis induction medium, i.e., a solution containing 1 mcM dexamethasone, 0.5 mM 3-Isobutyl-1-methylxanthine (IBMX), 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days (standard adipogenesis induction conditions). In yet other embodiments, each of CD34, CD39, and CD106 is expressed by less than 10% of the cells; less than 20% of the cells highly express CD56; and the cells do not differentiate into adipocytes, after incubation under the standard conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56; and the cells do not differentiate into adipocytes, under the standard conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days (modified adipogenic conditions). In still other embodiments, over 90% of the cells in each population do not differentiate into either adipocytes or osteocytes under the aforementioned standard conditions. In yet other embodiments, over 90% of the cells in each population do not differentiate into either adipocytes or osteocytes under the modified conditions. The aforementioned solutions will typically contain cell culture medium such as DMEM +10% serum or the like, as will be appreciated by those skilled in the art. In various embodiments, the cell population may be less than 50%, less than 40%, less than 30%, less than 20%, or less than 10%, or less than 5% positive for CD200. In other embodiments, the cell population is more than 50%, 60%, 70%, 80%, 90%, 95%, 97%, 98%, 99%, or 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetal tissue-derived placental cell populations that were expanded on 3D substrates.

In still other embodiments, the described ASC possess any other marker phenotype, other characteristic (e.g. secretion of factor(s), differentiation capability, resistance to differentiation, inhibition of T-cell proliferation, or stimulation of myoblast proliferation), or combination thereof that is mentioned and/or described in international patent application publ. no. WO 2019/239295, filed Jun. 10, 2019, to Zami Aberman et al, which is incorporated herein by reference.

In still other embodiments, the cells may be allogeneic, or in other embodiments, the cells may be autologous. In other embodiments, the cells may be fresh or, in other embodiments, frozen (for example, cryo-preserved).

In certain embodiments, any of the aforementioned ASC populations are used in the described methods and compositions. In other embodiments, CM obtained from the cells are used in the described methods and compositions. Each population may be freely combined with each of the described treatments, and each combination represents a separate embodiment. Furthermore, the cells utilized to generate CM or contained in the composition can be, in various embodiments, autologous, allogeneic, or xenogenic to the treated subject. Each type of cell may be freely combined with the therapeutic embodiments mentioned herein.

### Additional Method Characteristics for Preparation of ASC and CM Derived Therefrom

In some embodiments, the described placental ASC have been incubated in a 3D bioreactor. Each described embodiment for cell expansion may be combined with any of the described embodiments for therapeutic uses of ASC and CM derived therefrom.

In some embodiments, the described ASC or CM are/is harvested from a 3D bioreactor in which the ASC have been incubated. Alternatively or in addition, the cells are cryopreserved, and then are thawed, after which the cells are further expanded and/or CM are isolated therefrom. In other embodiments, after thawing, the cells are cultured in 2D culture, from which the ASC are isolated.

In certain embodiments, the described ASC are, or have been, subject to a 3D incubation, as described further herein. In more specific embodiments, the ASC have been incubated in a 2D adherent-cell culture apparatus, prior to the step of 3D culturing.

The terms “two-dimensional culture” and “2D culture” refer to a culture in which the cells are exposed to conditions that are compatible with cell growth and allow the cells to grow in a monolayer. An apparatus suitable for such growth is referred to as a “2D culture apparatus”. Such apparatuses will typically have flat growth surfaces (also referred to as a “two-dimensional substrate(s)” or “2D substrate(s)”), in some embodiments comprising an adherent material, which may be flat or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture” and “3D culture” refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface (also referred to as a “three-dimensional substrate” or “3D substrate”), in some embodiments comprising an adherent material, which is present in the 3D culture apparatus, e.g. the bioreactor. Certain, non-limiting embodiments of 3D culturing conditions suitable for expansion of adherent stromal cells are described in PCT Application Publ. No. WO/2007/108003, which is fully incorporated herein by reference in its entirety.

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or any type of fibrous matrix.

In still other embodiments, the described ASC are, or have been, subject to culturing conditions (e.g,. a growth substate, incubation time, bioreactor, seeding density, or harvest density) mentioned in international patent application publ. no. WO 2019/239295, filed Jun. 10, 2019, to Zami Aberman et al, which is incorporated herein by reference.

In other embodiments, the length of 3D culturing is at least 4 days; between 4-12 days; in other embodiments between 4-11 days; 4-10 days; 4-9 days; 5-9 days; 5-8 days; 6-8 days; or 5-7 days. In other embodiments, the 3D culturing is performed for 5-15 cell doublings, in other embodiments 5-14, 5-13, 5-12, 5-11, 5-10, 6-15, 6-14, 6-13, 6-12, 6-11, or 6-10 doublings.

In certain embodiments, 3D culturing can be performed in a 3D bioreactor. In some embodiments, the 3D bioreactor comprises a container for holding medium and a 3D attachment substrate disposed therein, and a control apparatus, for controlling pH, temperature, and oxygen levels and optionally other parameters. The terms attachment substrate and growth substrate are interchangeable.

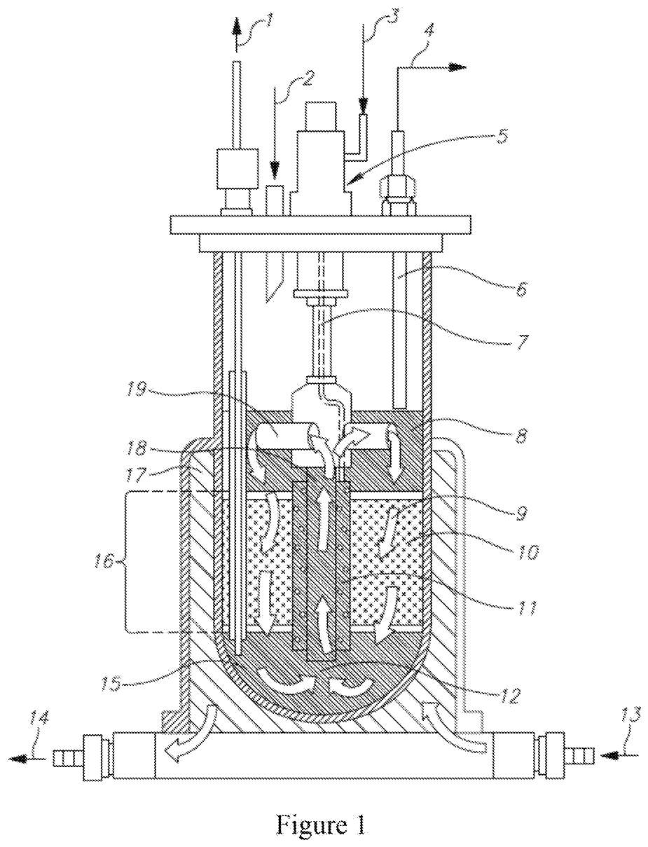

An exemplary, non-limiting bioreactor, the Celligen 310 Bioreactor, is depicted in FIG. 1. A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow stiffing initial rate is used to promote cell attachment, then agitation is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In still other embodiments, the matrix is a plug flow bioreactor which is, in other embodiments, packed with Fibra-cel® carriers (or, in other embodiments, other carriers).

In other embodiments, prefabricated or rigid scaffolds are utilized. Such scaffolds require, in some embodiments, migration of cells into the scaffold, after cell seeding. In other embodiments, physically crosslinked scaffolds may be utilized, which are, in further embodiments, gels that are formed via reversible changes in pH or temperature.

In other embodiments, microencapsulation is utilized. In certain embodiments, cells are immobilized within a semi-permeable material, e.g., a membrane that allows the diffusion of nutrients, oxygen, and growth factors essential for cell growth.

In more particular embodiments, cells may be removed from a 3D matrix while the matrix remains within the bioreactor. In certain embodiments, at least about 10%, 20%, or 30% of the cells are in the S and G2/M phases (collectively), at the time of harvest from the bioreactor.

In certain embodiments, the harvesting process comprises vibration or agitation, for example as described in PCT International Application Publ. No. WO 2012/140519, which is incorporated herein by reference. In certain embodiments, during harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator.

Those skilled in the art will appreciate that a variety of isotonic buffers may be used for washing cells and similar uses. Hank's Balanced Salt Solution (MSS; Life Technologies) is only one of many buffers that may be used.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

Effective dosages and schedules for administering the compositions may be determined empirically, and making such determinations is within the skill in the art. The dosage can be adjusted by the individual physician in the event of any counterindications. A typical dosage of the ASC ranges, in some embodiments, from ˜10 million to ˜500 million cells per administration, depending on the factors mentioned above. For example, the dosage of ASC can be 10, 20, 30, 40, 50, 60, 70, 80, 90, 100, 125, 150, 175, 200, 225, 250, 275, 300, 325, 350, 375, 400, 425, 450, 475, or 500 million cells or any amount in between. It is further understood that a range of ASC can be used including from ˜100 to ˜400 million cells, from ˜150 to ˜300 million cells. Accordingly, disclosed herein are therapeutic methods, wherein the dosage of ASC administered to the subject is 10, 20, 30, 40, 50, 60, 70, 80, 90, 100, 125, 150, 175, 200, 225, 250, 275, 300, 325, 350, 375, 400, 425, 450, 475, or 500 million cells or from 150 million-300 million cells. ASC, compositions comprising ASC, and/or medicaments manufactured using said ASC can be administered in a single dose, 2 doses, 3 doses, 2-5 doses, 2-10 doses, 1-10 doses, or 1-3 doses, over a time period of 1, 2, 3-6, 6-12, 2-12, 2-20, 3-20, or 4-20 weeks; or, in other embodiments, 2 3, 4, 5, 6, 7, 8, 9, 10, 11, or 12 months; or, in other embodiments, 1.5, 2, 3, 4, 5 years, or more.

In certain embodiments, the described pharmaceutical composition contains between 100-600 million ASC, for an adult subject. In other embodiments, the pharmaceutical composition contains between 100-400 million, 100-500 million, 150-600 million, 150-500 million, 150-400 million, 200-600 million, 200-500 million, or 200-400 million ASC, for an adult subject. In still other embodiments, the composition contains between 1.5-6 million ASC per kilogram, e.g., for a pediatric subject. In yet other embodiments, e.g., for a pediatric subject, the composition contains between 1.5-5 million, 1.5-4 million, 2-5 million, 2-4 million, 3-6 million, or 3-5 million ASC per kilogram. In certain embodiments, the administration is intramuscular. The exact formulation, route of administration and dosage can be, in some embodiments, chosen by the individual physician in view of the patient's condition.

Depending on the severity and responsiveness of the condition to be treated, dosing can be of a single or, in other embodiments, a plurality of administrations, with a course of treatment lasting from 2 days to 3 weeks or, in other embodiments, from 3 weeks to 3 months, or, in other embodiments, until alleviation of the disease state is achieved.

In certain embodiments, following administration, the majority of the cells, in other embodiments more than 60%, more than 70%, more than 80%, more than 90%, more than 95%, more than 96%, more than 97%, more than 98%, or more than 99% of the cells are no longer detectable within the subject 1 month after administration.

### Formulations

In some embodiments, the described composition is an injectable composition that is manufactured by adding 1 or more excipients, e.g. stabilizers and aqueous buffers, to placental ASC or CM thereof.

In other embodiments, the ASC are washed to remove serum present therewith. In more specific embodiments, xenogenic serum components are reduced by at least 90%, 95%, 99%, 99.5%, 99.8%, or 99.9%; or, in other embodiments, are undetectable by standard methods, e.g. mass spectrometry.

### Pharmaceutical Carriers

In certain embodiments, the described compositions comprise one or more pharmaceutically acceptable carriers. Herein, the term “pharmaceutically acceptable carrier” refers to a carrier or a diluent. In some embodiments, a pharmaceutically acceptable carrier does not cause significant irritation to a subject. In some embodiments, a pharmaceutically acceptable carrier does not abrogate the biological activity and properties of administered cells. Examples, without limitations, of carriers are propylene glycol, saline, emulsions, and mixtures of organic solvents with water. In some embodiments, the pharmaceutical carrier is an aqueous solution of saline.

In other embodiments, the composition further comprises at least one constituent to facilitate formulation, stability, and/or topical application of the composition. In more specific embodiments, the constituent comprises a flow regulating agent, a filler, an excipient, an alcohol, a preservative, a suspending agent, a stabilizer, a surfactant, an oil phase, an aqueous phase, a humectant, or a thickener. In other embodiments, the at least one additional constituent comprises colloidal silica, titanium dioxide, isopropyl alcohol, benzalkonium chloride, stearic acid, cetyl alcohol, isopropyl palmitate, methyparaben, propylparaben, sorbitan monostearate, sorbitol, polysorbate, milk, coconut oil, almond oil, lanolin, lecithin, or beeswax. In other embodiments, the described composition is a gel. In other embodiments, the composition is a lotion.

In still other embodiments, the composition comprises placental ASC in combination with an excipient selected from an osmoprotectant or cryoprotectant, an agent that protects cells from the damaging effect of freezing and ice formation. In certain embodiments, the cryoprotectant is a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin In still other embodiments, both an osmoprotectant and a carrier protein are present; in certain embodiments, the osmoprotectant and carrier protein may be the same compound. Alternatively or in addition, the composition is frozen. The cells may be any embodiment of ASC mentioned herein, each of which represents a separate embodiment. In more specific embodiments, DMSO is present at a concentration of 2-5%; or, in other embodiments, 5-10%; or, in other embodiments, 2-10%, 3-5%, 4-6%; 5-7%, 6-8%, 7-9%, 8-10%. DMSO, in other embodiments, is present with a carrier protein, a non-limiting example of which is albumin, e.g. human serum albumin (HSA). In certain embodiments, HSA is present at 2-10%, 3-10%, 4-10%, 5-10%, 2-9%, 2-8%, 3-7%, 4-6%, 4.5-5.5%, or 5% (weight per volume). In still other embodiments, DMSO and HSA are both present in a saline solution (a non-limiting example of which is Plasma-Lyte® A (commercially available from Baxter).

In other embodiments, for injection, the described ASC or other active ingredients may be formulated in aqueous solutions, e.g. in a physiologically compatible buffer, non-limiting examples of which are Hank's solution, Ringer's solution, and a physiological salt buffer.

### Routes

In certain embodiments, the described pharmaceutical compositions are administered intramuscularly. In other embodiments, the composition is administered systemically. Alternatively, the composition is administered locally, for example, via injection of the pharmaceutical composition directly into an exposed or affected tissue region of a patient. In other embodiments, the composition is administered intravenously (IV), subcutaneously (SC), or intraperitoneally (IP), each of which is considered a separate embodiment. In this regard, “intramuscular” administration refers to administration into the muscle tissue of a subject; “subcutaneous” to administration just below the skin; “intravenous” to administration into a vein of a subject; and “intraperitoneal” refers to administration into the peritoneum of a subject. In still other embodiments, the cells are administered intratracheally, intrathecally, by inhalation, or intranasally. In certain embodiments, lung-targeting routes of administration are used. In certain embodiments, such routes utilize cells encapsulated in liposomes (or, in some embodiments, other physical barriers) to reduce entrapment within the lungs.

In various embodiments, the described ASC are administered to the subject within 1, 2, 3, 4, 6, 8, 10, 12, 15, 18, 24, 30, 36, or 48 hours; or within 3, 4, 5, 6, 8, 10, 12, or 20 days of diagnosis (or, in other embodiments, onset) of any of the herein-described conditions (each of which represents a separate embodiment.). In more specific embodiments, the described compositions are administered 1-24, 2-24, 3-24, 4-24, 5-24, 6-24, 8-24, 10-24, 12-48, 1-48, 2-48, 3-48, 4-48, 5-48, 6-48, 8-48, 10-48, 12-48, 18-48, 24-48, 1-72, 2-72, 3-72, 4-72, 5-72, 6-72, 8-72, 10-72, 12-72, 18-72, 24-72, or 36-72 hours after onset of any of the herein-described conditions. In still other embodiments, the described compositions are administered 3-48, 4-48, 5-48, or 6-48 hours after an onset of any of the herein-described conditions.

In various embodiments, engraftment of the described cells in the host is not required for the cells to exert the described therapeutic effects, each of which is considered a separate embodiment. In other embodiments, engraftment is required for the cells to exert the effect(s). For example, the cells may, in various embodiments, be able to exert a therapeutic effect, without themselves surviving for more than 3 days, more than 4 days, more than 5 days, more than 6 days, more than 7 days, more than 8 days, more than 9 days, more than 10 days, or more than 14 days after administration.

Compositions including the described preparations formulated in a compatible pharmaceutical carrier may also be prepared, placed in an appropriate container, and labeled for treatment of an indicated condition.

It is clarified that each embodiment of the described ASC or CM may be freely combined with each embodiment relating to a therapeutic method or pharmaceutical composition.

### Subjects

In certain embodiments, the subject treated by the described methods and compositions is a human In certain embodiments, the subject has a viral infection. In still other embodiments, the subject has ARDS, pulmonary hypertension, lung fibrosis, acute kidney injury, gastrointestinal injury, or another complication of a viral infection. In some embodiments, the subject is male. In other embodiments, the subject is female. In some embodiments, the subject is at increased risk of complications of COVID-19. In certain embodiments, the subject is an elderly subject, for example a subject over 75, or in other embodiments, over 65, over 70, 75, over 80, 70-85, 75-85, or 75-90 years in age. In other embodiments, the subject has asthma. In still other embodiments, the subject has chronic lung disease. In yet other embodiments, the subject has heart disease. In yet other embodiments, the subject is immunocompromised. In yet other embodiments, the subject has chronic kidney disease and/or is undergoing dialysis. In yet other embodiments, the subject has liver disease. In other embodiments, the subject is severely obese (e.g., has a body mass index [BMI] of 40 or higher). In other embodiments, the subject has hypercytokinemia, SIRS, or another type of hyperactive immune response.

In yet other embodiments, the subject is a pediatric subject, for example a subject under 18, under 15, under 12, under 10, under 8, under 6, under 5, under 4, under 3, or under 2 years, or under 18, 15, 12, 10, 8, 6, 5, 4, 3, 2, or 1 month in age; or is an adult subject, for example ages 18-60, 18-55, 18-50, 20-60, 20-55, 20-50, 20-45, 20-40, 20-35, 20-30, 25-60, 30-60, 40-60, or 50-60.

In other embodiments, the subject is an animal. In some embodiments, treated animals include domesticated animals and laboratory animals, e.g., non-mammals and mammals, for example non-human primates, rodents, pigs, dogs, and cats. In certain embodiments, the subject is administered with additional therapeutic agents or cells.

### Sequential Administration of ASC from Multiple Donors

In certain embodiments, the subject is administered: (a) a first pharmaceutical composition, comprising allogeneic placental ASC from a first donor; and subsequently (b) a second pharmaceutical composition comprising allogeneic placental ASC from a second donor, wherein the second donor differs from the first donor in at least one allele group of HLA-A or HLA-B.

In certain embodiments, each of the pharmaceutical compositions contains between 100-600 million ASC, for an adult subject. In other embodiments, the pharmaceutical compositions each contain between 100-400 million, 100-500 million, 150-600 million, 150-500 million, 150-400 million, 200-600 million, 200-500 million, or 200-400 million ASC, for an adult subject. In still other embodiments, the compositions each contain between 1.5-6 million ASC per kilogram, e.g., for a pediatric subject. In yet other embodiments, e.g., for a pediatric subject, the compositions each contain between 1.5-5 million, 1.5-4 million, 2-5 million, 2-4 million, 3-6 million, or 3-5 million ASC per kilogram. In certain embodiments, the administration is intramuscular.

Reference to ASC “from” or “derived from” a donor is intended to encompass cells removed from or otherwise obtained from the donor, followed by optional steps of ex-vivo cell culture, expansion, and/or other treatments to improve the therapeutic efficacy of the cells; and/or combination with pharmaceutical excipients. Those skilled in the art will appreciate that the aforementioned optional steps will not alter the HLA genotype of the ASC, absent intentional modification of the HLA genotype (e.g., using CRISPR-mediating editing or the like). Cell populations with an intentionally modified HLA genotype are not intended to be encompassed. ASC populations that contain a mixture of cells from more than one donor are also not intended to be encompassed.

Reference to a second donor “differ/differs/differing” from a first donor in at least one allele group of HLA-A or HLA-B denotes that the DNA of the second donor comprises at least one HLA-A or HLA-B allele belonging to an allele group not represented in the alleles of the first donor. (Typically [except in the case of a homozygous first donor], the DNA of the first donor will also comprise at least one HLA-A or HLA-B allele belonging to an allele group not represented in the alleles of the second donor). Similarly, a second donor “differs from” a first donor in at least one allele supertype if the DNA of the second donor comprises at least one HLA-A or HLA-B allele belonging to a supertype not represented in the alleles of the first donor. These terms are intended to be used analogously in various contexts herein, except where indicated otherwise.

In other embodiments, the second donor in the described therapeutic methods and compositions differs from the first donor in at least one allele group of HLA-A. In still other embodiments, the second donor differs from the first donor in at least one allele group of HLA-B.

In yet other embodiments, the second donor differs from the first donor in at least two HLA-A allele groups of or, in other embodiments, in at least 2 HLA-B allele groups; or, in other embodiments, at least one allele group of each of HLA-A and HLA-B.

In other embodiments, the second donor differs from the first donor in at least one HLA-A allele supertype or, in other embodiments, at least one HLA-B allele supertype.

In still other embodiments, the second donor differs from the first donor in at least two allele supertypes of HLA-A or HLA-B, which may be, in more specific embodiments, an HLA-A allele supertype, an HLA-B allele supertype, or a combination thereof.

Alternatively or in addition, the second donor differs from the first donor in at least one allele group of HLA-DR, or in other embodiments, in 2 HLA-DR allele groups.

Further embodiments of dosing regimens are described in WO 2019/239295, in the name of Zami Aberman et al., which is incorporated herein by reference.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including ASC. In another aspect, the kits and articles of manufacture comprise a label, instructions, and packaging material, for example for treating a disorder or therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1: Culturing and Production of Adherent Placental Cells

Placenta-derived cell populations containing over 90% maternal tissue-derived cells were prepared as described in Example 1 of International Patent Application WO 2016/098061, which is incorporated herein by reference in its entirety. The cell expansion and harvesting process consisted of 3 stages, followed by downstream processing steps: Stage 1, the intermediate cell stock (ICS) production; Stage 2, the thawing of the ICS and initial further culture steps; and Stage 3, additional culture steps, first in tissue culture dishes, and then on Fibra-Cel® carriers in a bioreactor. All steps were performed in the presence of serum-containing medium. The downstream processing steps included harvest from flasks or bioreactor/s, cell concentration, washing, formulation, filling and cryopreservation. The procedure included periodic testing of the growth medium for sterility and contamination, all as described in international patent application publ. no. WO 2019/239295, which is incorporated herein by reference.

### Example 2: Culture oF Placental Cells in Serum-Free Medium

Methods

The cell harvesting and expansion process consisted of 3 stages, followed by downstream processing steps: Stage 1, the intermediate cell stock (ICS) production; Stage 2, the thawing of the ICS and initial further culture steps; and Stage 3, additional culture steps, first in tissue culture dishes, and then on Fibra-Cel® carriers in a bioreactor. The downstream processing steps included harvest from flasks or bioreactor/s, cell concentration, washing, formulation, filling and cryopreservation. The procedure included periodic testing of the growth medium for sterility and contamination, all as described in international patent application publ. no. WO 2019/239295, which is incorporated herein by reference. Bone marrow migration assays were also performed as described in WO 2019/239295.

Results

Placental cells were extracted and expanded in serum-free (SF) medium for 3 passages. Cell characteristics of eight batches were assessed and were found to exhibit similar patterns of cell size and PDL (population doubling level since passage 1) as shown for a representative batch in Table 1. Cells also significantly enhanced hematopoiesis in a bone marrow migration (BMM) assay.

TABLE 1
Characteristics of placental cells expanded in SF medium.

| | | | Total | cell | |
|---|---|---|---|---|---|
| | | | growth | size | |
| BATCH | GROUP | Passage | (days) | (μm) | PDL |
| PD200114SFM | A | 1 | 8 | 20.3 | NA |
| | | 2 | 14 | 20.9 | 3.4 |
| | | 3 | 20 | 19.7 | 7 |
| | B | 1 | 8 | 19.5 | NA |
| | | 2 | 15 | 21.5 | 3.4 |
| | | 3 | 21 | 17 | 5.1 |

| | | | |
|---|---|---|---|
| Average P 3 | 19.1 | 17.55 | 6.12 |
| % CV P 3 | 8 | 9 | 11 |

### Example 3: Osteocyte and Adipocyte Differentiation Assays

ASC were prepared as described in Example 1. BM adherent cells were obtained as described in WO 2016/098061 to Esther Lukasiewicz Hagai and Rachel Ofir, which is incorporated herein by reference in its entirety. Osteogenesis and adipogenesis assays were performed as described in Example 2 of WO 2016/098061. Incubation of BM-derived adherent cells in osteogenic induction medium resulted in differentiation of over 50% of the BM cells, as demonstrated by positive alizarin red staining. On the contrary, none of the placental-derived cells exhibited signs of osteogenic differentiation. Next, a modified osteogenic medium comprising Vitamin D and higher concentrations of dexamethasone was used. Over 50% of the BM cells underwent differentiation into osteocytes, while none of the placental-derived cells exhibited signs of osteogenic differentiation.

Adipocyte induction. Adipocyte differentiation of placenta- or BM-derived adherent cells in adipocyte induction medium resulted in differentiation of over 50% of the BM-derived cells, as demonstrated by positive oil red staining and by typical morphological changes (e.g., accumulation of oil droplets in the cytoplasm). In contrast, none of the placental-derived cells differentiated into adipocytes. Next, a modified medium containing a higher indomethacin concentration was used. Over 50% of the BM-derived cells underwent differentiation into adipocytes. In contrast, none of the placental-derived cells exhibited morphological changes typical of adipocytes.

### Example 4: Further Osteocyte and Adipocyte Differentiation Assays

ASC were prepared as described in Example 2. Adipogenesis and Osteogenesis were assessed using the STEMPRO® Adipogenesis Differentiation Kit (GIBCO, Cat #A1007001) and the STEMPRO® Osteogenesis Differentiation Kit (GIBCO, Cat #A1007201), respectively.

Results

Adipogenesis and Osteogenesis of placental cells grown in SRM (3 different batches) or in full DMEM were tested. In adipogenesis assays, BM-MSCs treated with differentiation medium stained positively with Oil Red O. By contrast, 2/3 of the SRM batches exhibited negligible staining, and the other SRM batch, as well as the full DMEM-grown cells, did not exhibit any staining at all, showing that they lacked significant adipogenic potential. In osteogenesis assays, BM-MSCs treated with differentiation medium stained positively with Alizarin Red S. By contrast, none of the placental cell batches grown in SRM or full DMEM exhibited staining, showing that they lacked significant osteogenic potential.

### Example 5: Studies of Factors Secreted by Placental ASC

CM was prepared from two batches each of maternal ASC, fetal ASC expanded in serum-containing medium, and fetal ASC expanded in SFM, after a 6-day bioreactor incubation; or a 2-day incubation in plates, changing the medium once per day.

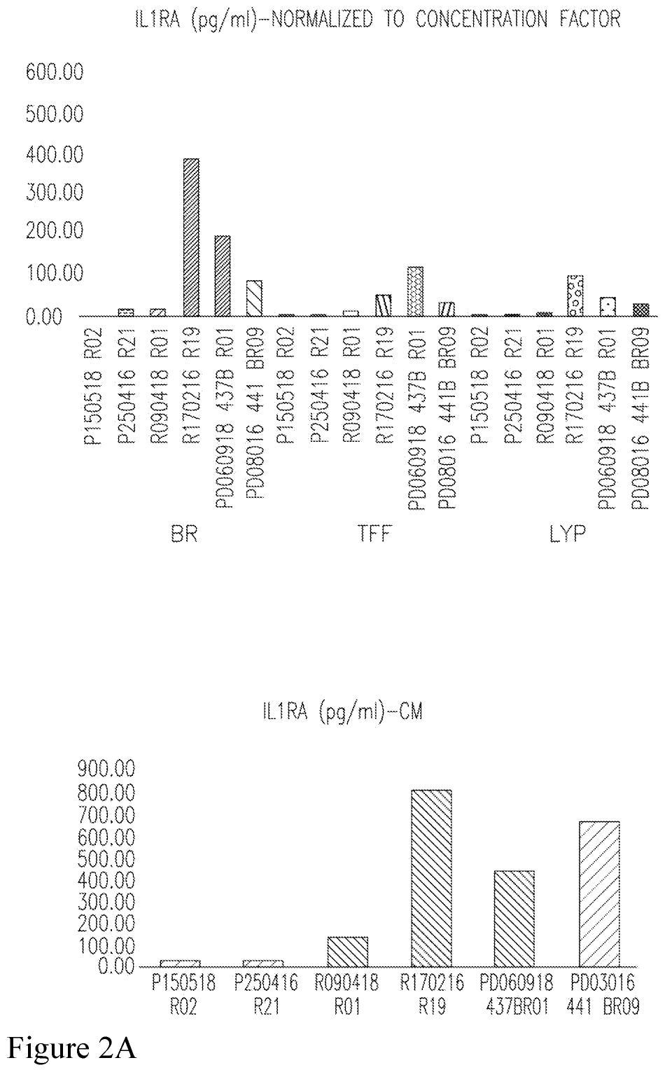

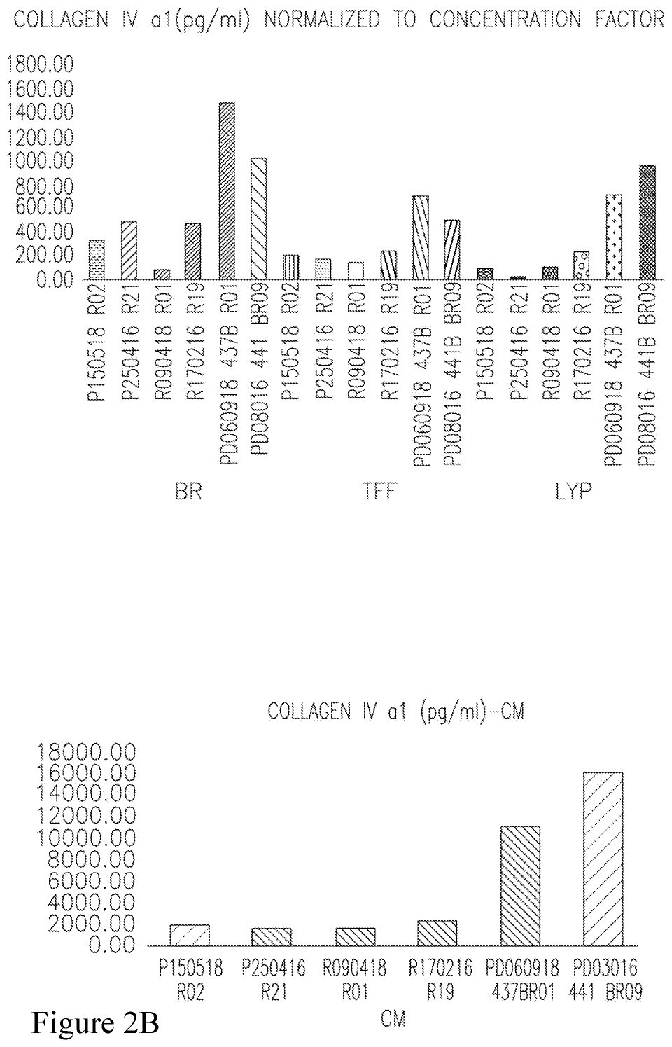

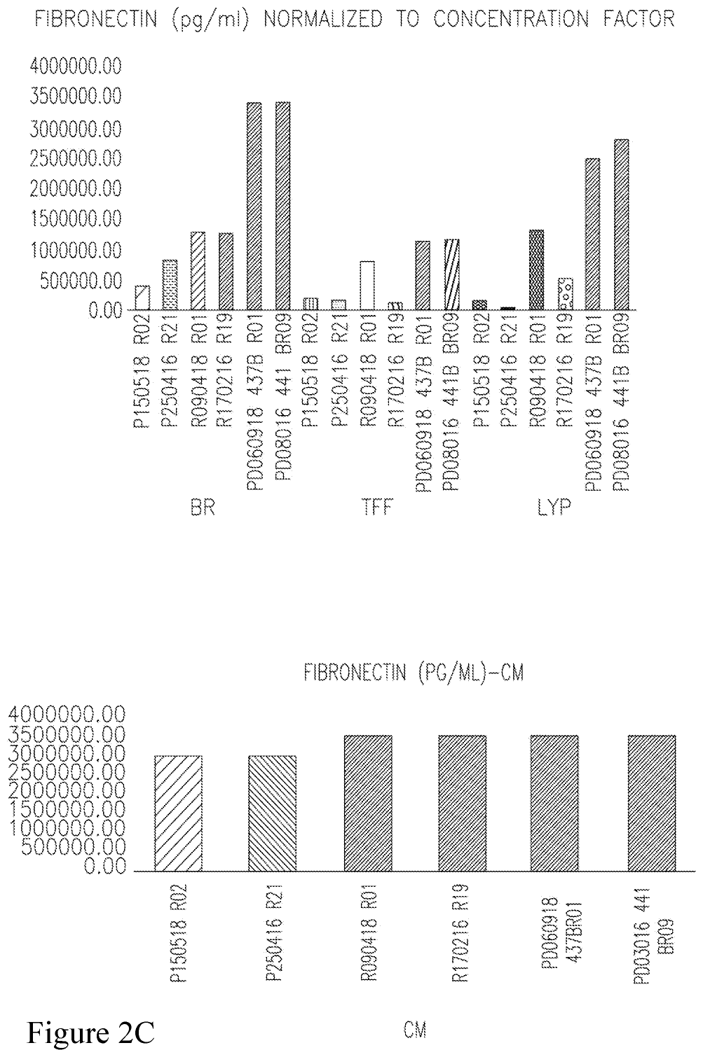

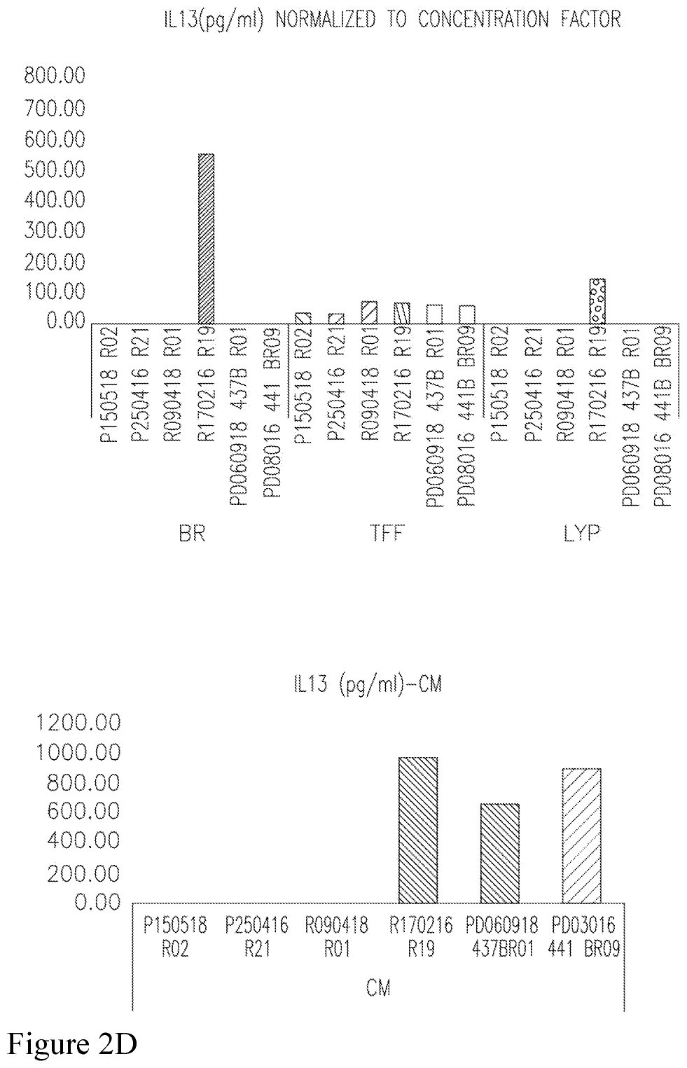

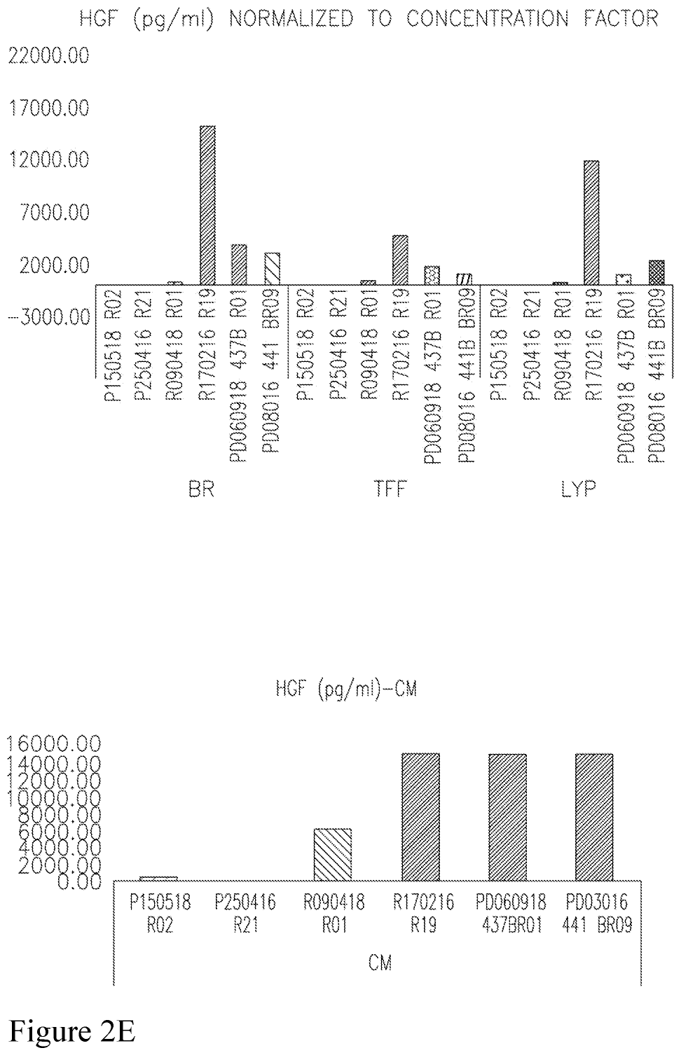

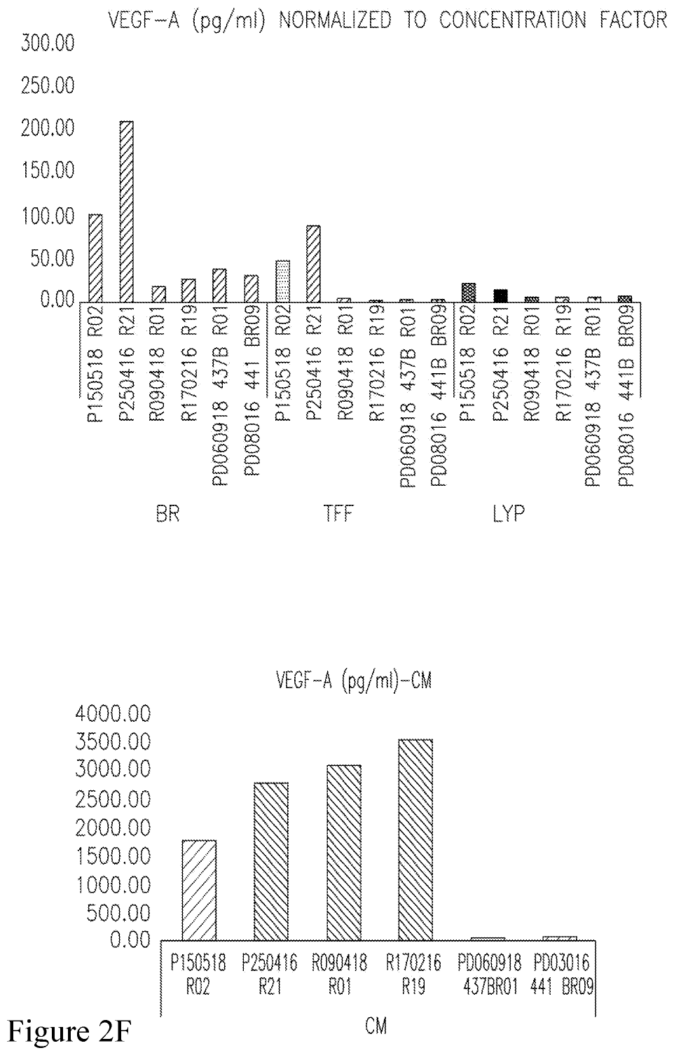

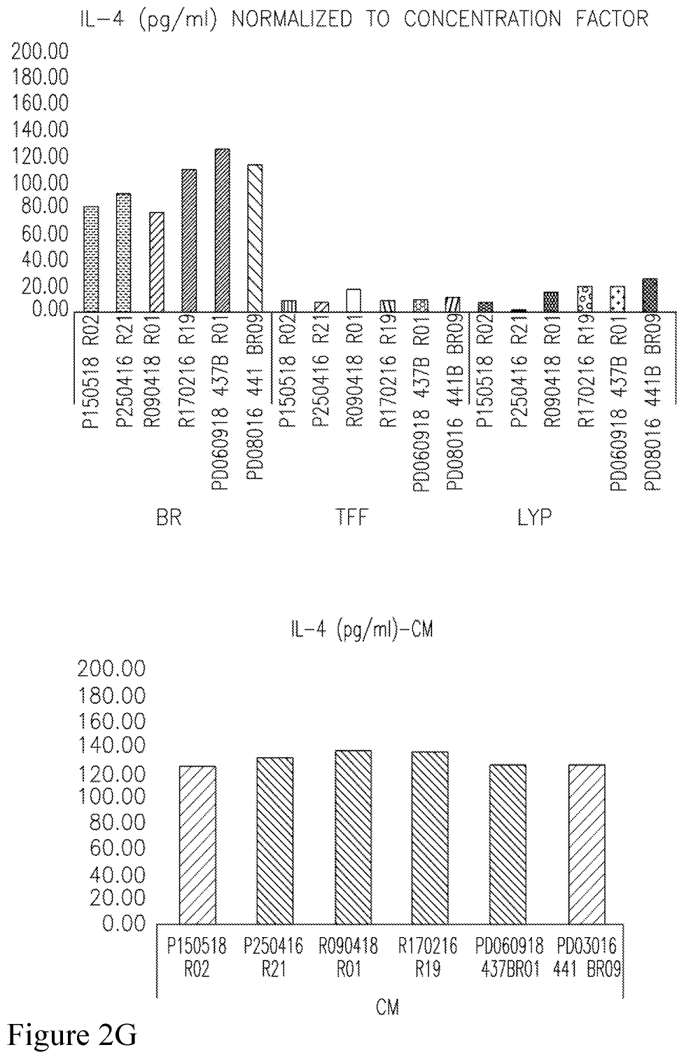

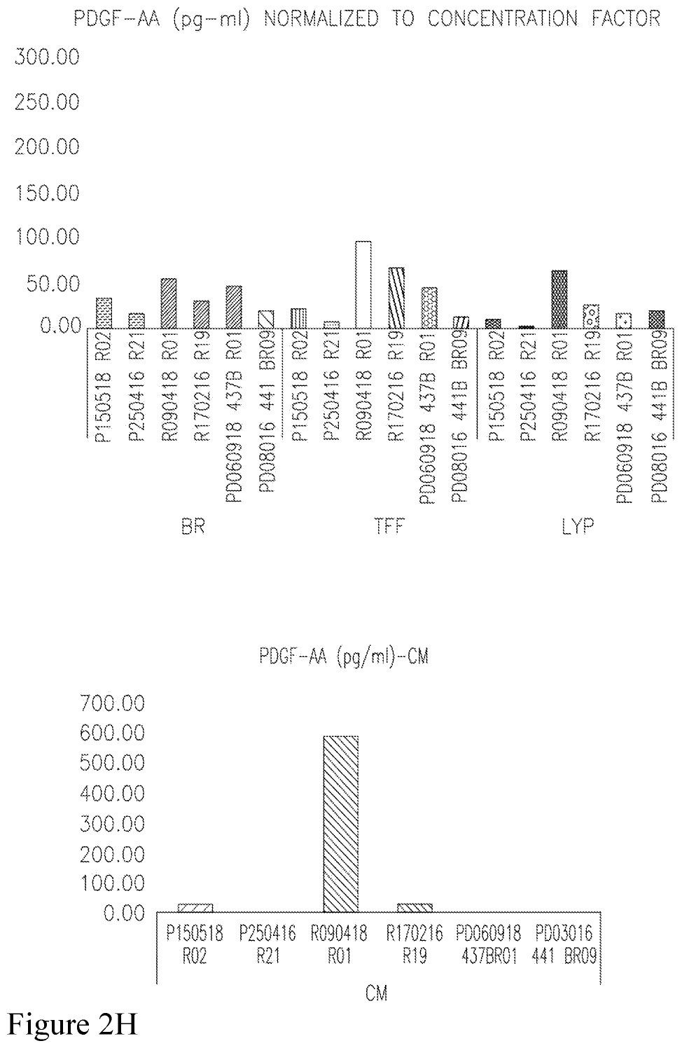

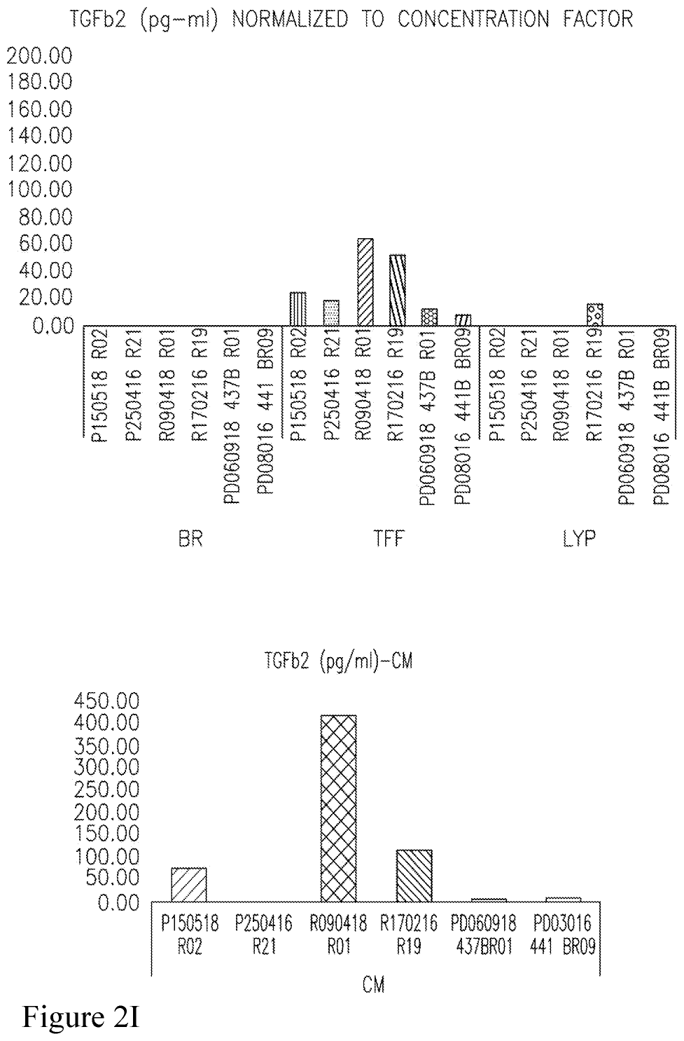

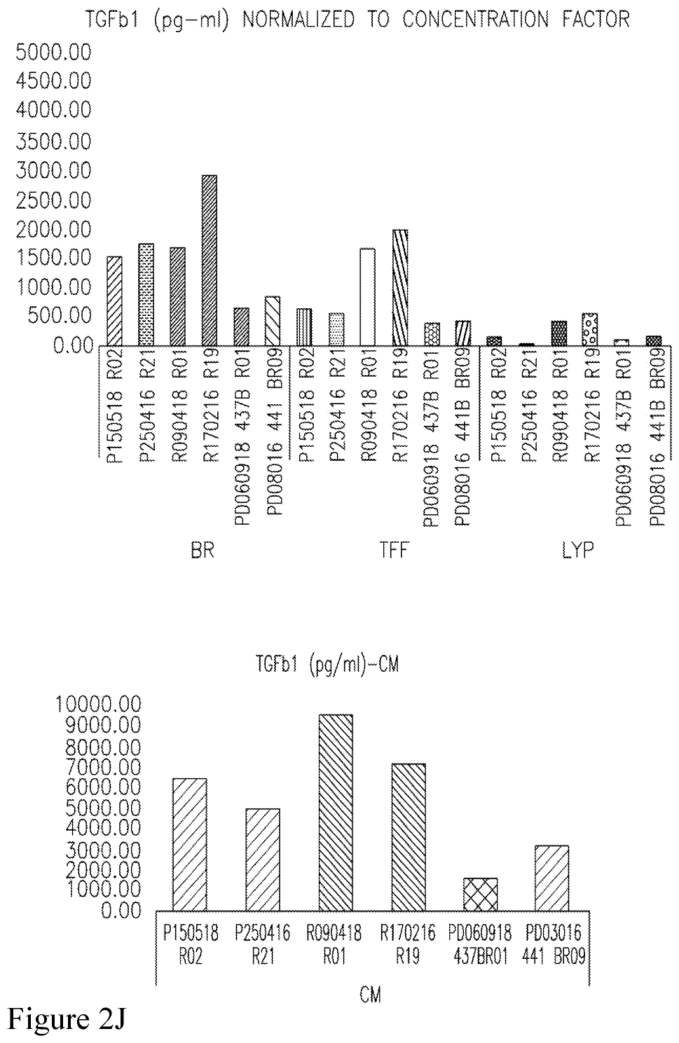

Secreted protein expression was measured by Luminex®. (D. Collagen 1-alpha were highly expressed in all samples. IL-1-ra, Collagen IV-la, Fibronectin, IL-13, HGF, VEGF-A, IL-4, PDGF-AA, TIMP-1, TGFb2, TGFb1 were all significantly expressed in at least some samples, while IL-16 was expressed at negligible or no level (FIGS. 2A-J and Tables 2-3).

TABLE 2
summarizes protein expression of the indicated
proteins in bioreactor media.

| Protein | Maternal | Fetal/serum | Fetal/SF |
|---|---|---|---|
| Collagen 1a | +++ | +++ | +++ |
| IL-10 | − | − | − |
| EGF | − | − | − |
| IL-1RA | − | ++ | ++ |
| bFGF | − | − | ++ |
| Collagen IVa1 | ++ | ++ | +++ |
| Fibronectin | +++ | +++ | +++ |
| IL-13 | + | ++ | + |
| HGF | − | +++ | +++ |
| MMP-1 | +++ | +++ | +++ |
| MMP-2 | +++ | +++ | +++ |
| IL-16 | + | + | + |
| VEGF-A | ++ | + | + |
| IL-4 | | + | + |
| PDGF-AA | + | + | + |
| TIMP1 | +++ | +++ | +++ |
| TGFb3 | − | − | − |
| TGFb2 | + | + | + |
| TGFb1 | +++ | +++ | +++ |

−, +, ++, and +++ indicate <10, 10-100, 100-1000, and >1000 pg/ml, respectively.

Mass spectrometry was performed on fetal/placental ASC-CM from a bioreactor incubation, and tryptic peptides of human origin were identified by their sequences. The peptides are shown in Table 3.

TABLE 3
Tryptic peptides from placental ASC-CM. “HS” refers to Homo sapiens.

| Protein name | gene name (indicated after “GN”) | Uniprot name (in square brackets) |
|---|---|---|
| Z | Z | Z |

Alpha-2-macroglobulin OS = HS GN = A2M PE = 1 SV = 3 - [A2MG_HUMAN]
Agrin OS = HS GN = AGRN PE = 1 SV = 5 - [AGRIN_HUMAN]
Serum albumin OS = HS GN = ALB PE = 1 SV = 1 - [A0A0C4DGB6_HUMAN]
Annexin A1 OS = HS GN = ANXA1 PE = 1 SV = 2 - [ANXA1_HUMAN]
Annexin (Fragment) OS = HS GN = ANXA2 PE = 1 SV = 1 - [H0YMM1_HUMAN]
APOC2 protein OS = HS GN = APOC2 PE = 1 SV = 1 - [Q6P163_HUMAN]
Actin-related protein 2/3 complex subunit 2 OS = HS GN = ARPC2 PE = 1 SV = 1 - [ARPC2_HUMAN]
Renin receptor (Fragment) OS = HS GN = ATP6AP2 PE = 1 SV = 1 - [A0A1B0GWD6_HUMAN]
Beta-2-microglobulin (Fragment) OS = HS GN = B2M PE = 1 SV = 1 - [H0YLF3_HUMAN]
Beta-1,4-glucuronyltransferase 1 OS = HS GN = B4GAT1 PE = 1 SV = 1 - [B4GA1_HUMAN]
Bone morphogenetic protein 1 OS = HS GN = BMP1 PE = 1 SV = 2 - [BMP1_HUMAN]
Complement C4-A OS = HS GN = C4A PE = 1 SV = 1 - [A0A0G2JPR0_HUMAN]
Calcium-binding protein 39-like OS = HS GN = CAB39L PE = 1 SV = 1 - [B7ZBJ4_HUMAN]
Cell adhesion molecule 1 OS = HS GN = CADM1 PE = 1 SV = 1 - [A0A087X0T8_HUMAN]
Capping protein (Actin filament) muscle Z-line, beta, isoform CRA_a OS = HS GN = CAPZB PE = 1
SV = 1 - [B1AK87_HUMAN]
CD44 antigen OS = HS GN = CD44 PE = 1 SV = 2 - [HOYD13_HUMAN]
Tetraspanin OS = HS GN = CD81 PE = 1 SV = 1 - [E9PJK1_HUMAN]
Cadherin-2 OS = HS GN = CDH2 PE = 1 SV = 4 - [CADH2_HUMAN]
Chymotrypsin-like elastase family member 1 OS = HS GN = CELA1 PE = 1 SV = 2 - [CELA1_HUMAN]
Collagen alpha-1(XI) chain (Fragment) OS = HS GN = COL11A1 PE = 1 SV = 8 - [C9JMN2_HUMAN]
Collagen alpha-1(XII) chain OS = HS GN = COL12A1 PE = 1 SV = 1 - [D6RGG3_HUMAN]
Collagen alpha-1(I) chain OS = HS GN = COL1A1 PE = 1 SV = 5 - [CO1A1_HUMAN]
Collagen alpha-1(III) chain OS = HS GN = COL3A1 PE = 1 SV = 4 - [CO3A1_HUMAN]
Collagen alpha-1(IV) chain OS = HS GN = COL4A1 PE = 1 SV = 3 - [CO4A1_HUMAN]
Collagen alpha-2(IV) chain OS = HS GN = COL4A2 PE = 1 SV = 4 - [CO4A2_HUMAN]
Collagen alpha-1(VI) chain OS = HS GN = COL6A1 PE = 1 SV = 1 - [A0A087X0S5_HUMAN]
Collagen alpha-3(VI) chain OS = HS GN = COL6A3 PE = 1 SV = 5 - [CO6A3_HUMAN]
Ceruloplasmin OS = HS GN = CP PE = 1 SV = 1 - [CERU_HUMAN]
Cystatin-C OS = HS GN = CST3 PE = 1 SV = 1 - [CYTC_HUMAN]
Connective tissue growth factor OS = HS GN = CTGF PE = 1 SV = 2 - [CTGF_HUMAN]
Cathepsin Z OS = HS GN = CTSZ PE = 1 SV = 1 - [CATZ_HUMAN]
Protein CutA OS = HS GN = CUTA PE = 1 SV = 1 - [C9IZG4_HUMAN]
Stromal cell-derived factor 1 OS = HS GN = CXCL12 PE = 1 SV = 1 - [SDF1_HUMAN]
Cytoplasmic FMR1-interacting protein 1 OS = HS GN = CYFIP1 PE = 1 SV = 1 - [CYFP1_HUMAN]
Protein CYR61 OS = HS GN = CYR61 PE = 1 SV = 1 - [CYR61_HUMAN]
Dermcidin OS = HS GN = DCD PE = 1 SV = 2 - [DCD_HUMAN]
Dickkopf-related protein 1 OS = HS GN = DKK1 PE = 1 SV = 1 - [DKK1_HUMAN]
Desmoglein-1 OS = HS GN = DSG1 PE = 1 SV = 2 - [DSG1_HUMAN]
Desmoplakin OS = HS GN = DSP PE = 1 SV = 3 - [DESP_HUMAN]
EF-hand domain-containing protein D2 OS = HS GN = EFHD2 PE = 1 SV = 1 - [EFHD2_HUMAN]
Eukaryotic translation initiation factor 4 gamma 1 (Fragment) OS = HS GN = EIF4G1 PE = 1 SV = 1 -
[C9J6B6_HUMAN]
Eukaryotic translation initiation factor 5A OS = HS GN = EIF5A2 PE = 1 SV = 1 - [F8WCJ1_HUMAN]
Fatty acid-binding protein, heart OS = HS GN = FABP3 PE = 1 SV = 1 - [S4R3A2_HUMAN]
Fibulin-1 OS = HS GN = FBLN1 PE = 1 SV = 1 - [B1AHL2_HUMAN]
Fibrillin-1 OS = HS GN = FBN1 PE = 1 SV = 3 - [FBN1_HUMAN]
Filamin-A OS = HS GN = FLNA PE = 1 SV = 1 - [Q5HY54_HUMAN]
Fibronectin OS = HS GN = FN1 PE = 1 SV = 4 - [FINC_HUMAN]
Follistatin-related protein 1 OS = HS GN = FSTL1 PE = 1 SV = 1 - [FSTL1_HUMAN]
Rab GDP dissociation inhibitor beta OS = HS GN = GDI2 PE = 1 SV = 2 - [GDIB_HUMAN]
Glypican-1 OS = HS GN = GPC1 PE = 1 SV = 2 - [H7C410_HUMAN]
Histone H3 OS = HS GN = H3F3B PE = 1 SV = 1 - [K7EMV3_HUMAN]
HCG1745306, isoform CRA_a OS = HS GN = HBA2 PE = 1 SV = 1 - [G3VIN2_HUMAN]
Hemoglobin subunit delta OS = HS GN = HBD PE = 1 SV = 2 - [HBD_HUMAN]
Hepatocyte growth factor activator OS = HS GN = HGFAC PE = 1 SV = 1 - [HGFA_HUMAN]
Histone H2A type 1-H OS = HS GN = HIST1H2AH PE = 1 SV = 3 - [H2A1H_HUMAN]
HLA class I histocompatibility antigen, Cw-6 alpha chain OS = HS GN = HLA-C PE = 1 SV = 1 -
[A0A140T9Z4_HUMAN]
Heterogeneous nuclear ribonucleoproteins A2/B1 OS = HS GN = HNRNPA2B1 PE = 1 SV = 1 -
[A0A087WUI2_HUMAN]
Hornerin OS = HS GN = HRNR PE = 1 SV = 2 - [HORN_HUMAN]
Heat shock protein HSP 90-alpha OS = HS GN = HSP90AA1 PE = 1 SV = 5 - [HS90A_HUMAN]
Endoplasmin OS = HS GN = HSP90B1 PE = 1 SV = 1 - [Q96GW1_HUMAN]
Heat shock 70 kDa protein 1B OS = HS GN = HSPA1B PE = 1 SV = 1 - [HS71B_HUMAN]
Heat shock cognate 71 kDa protein OS = HS GN = HSPA8 PE = 1 SV = 1 - [E9PKE3_HUMAN]
Basement membrane-specific heparan sulfate proteoglycan core protein OS = HS GN = HSPG2
PE = 1 SV = 4 - [PGBM_HUMAN]
Serine protease HTRA1 OS = HS GN = HTRA1 PE = 1 SV = 1 - [HTRA1_HUMAN]
E3 ubiquitin-protein ligase HUWE1 OS = HS GN = HUWE1 PE = 1 SV = 3 - [HUWE1_HUMAN]
Insulin-like growth factor-binding protein 5 OS = HS GN = IGFBP5 PE = 1 SV = 1 - [IBP5_HUMAN]
Insulin-like growth factor-binding protein 6 OS = HS GN = IGFBP6 PE = 1 SV = 1 - [IBP6_HUMAN]
Insulin-like growth factor-binding protein 7 OS = HS GN = IGFBP7 PE = 1 SV = 1 - [IBP7_HUMAN]
Insulin-like growth factor I (Fragment) OS = HS GN = IGF-I PE = 1 SV = 1 - [Q13429_HUMAN]
Junction plakoglobin OS = HS GN = JUP PE = 1 SV = 3 - [PLAK_HUMAN]
Keratinocyte proline-rich protein OS = HS GN = KPRP PE = 1 SV = 1 - [KPRP_HUMAN]
Laminin subunit alpha-1 OS = HS GN = LAMA1 PE = 1 SV = 2 - [LAMA1_HUMAN]
Laminin subunit alpha-4 OS = HS GN = LAMA4 PE = 1 SV = 1 - [A0A0A0MTC7_HUMAN]
Laminin subunit beta-1 OS = HS GN = LAMB1 PE = 1 SV = 2 - [LAMB1_HUMAN]
Laminin subunit gamma-1 OS = HS GN = LAMC1 PE = 1 SV = 3 - [LAMC1_HUMAN]
Galectin-1 OS = HS GN = LGALS1 PE = 1 SV = 2 - [LEG1_HUMAN]
Galectin-3 OS = HS GN = LGALS3 PE = 1 SV = 5 - [LEG3_HUMAN]
Galectin-3-binding protein OS = HS GN = LGALS3BP PE = 1 SV = 1 - [LG3BP_HUMAN]
LIM and senescent cell antigen-like-containing domain protein 1 OS = HS GN = LIMS1 PE = 1 SV = 4 -
[LIMS1_HUMAN]
Vesicular integral-membrane protein VIP36 OS = HS GN = LMAN2 PE = 1 SV = 1 - [LMAN2_HUMAN]
Protein-lysine 6-oxidase OS = HS GN = LOX PE = 1 SV = 2 - [LYOX_HUMAN]
Lysyl oxidase homolog 2 (Fragment) OS = HS GN = LOXL2 PE = 1 SV = 1 - [H0YAR1_HUMAN]
Latent-transforming growth factor beta-binding protein 2 OS = HS GN = LTBP2 PE = 1 SV = 1 -
[G3V3X5_HUMAN]
Lysozyme C OS = HS GN = LYZ PE = 1 SV = 1 - [LYSC_HUMAN]
72 kDa type IV collagenase OS = HS GN = MMP2 PE = 1 SV = 2 - [MMP2_HUMAN]
Moesin OS = HS GN = MSN PE = 1 SV = 3 - [MOES_HUMAN]
Metallothionein-1E OS = HS GN = MT1E PE = 1 SV = 1 - [MT1E_HUMAN]
Matrix-remodeling-associated protein 5 OS = HS GN = MXRA5 PE = 2 SV = 3 - [MXRA5_HUMAN]
Myosin-9 OS = HS GN = MYH9 PE = 1 SV = 4 - [MYH9_HUMAN]
Myosin light polypeptide 6 (Fragment) OS = HS GN = MYL6 PE = 1 SV = 1 - [F8VPF3_HUMAN]
Neurobeachin-like protein 2 OS = HS GN = NBEAL2 PE = 1 SV = 2 - [NBEL2_HUMAN]
Nidogen-1 OS = HS GN = NID1 PE = 1 SV = 3 - [NID1_HUMAN]
Epididymal secretory protein E1 (Fragment) OS = HS GN = NPC2 PE = 1 SV = 1 -
[G3V2V8_HUMAN]
Puromycin-sensitive aminopeptidase OS = HS GN = NPEPPS PE = 1 SV = 1 - [E9PLK3_HUMAN]
Nuclear transport factor 2 (Fragment) OS = HS GN = NUTF2 PE = 1 SV = 1 - [H3BRV9_HUMAN]
Ubiquitin thioesterase OTUB1 OS = HS GN = OTUB1 PE = 1 SV = 1 - [F5GYN4_HUMAN]
Beta-parvin OS = HS GN = PARVB PE = 1 SV = 1 - [A0A087WZB5_HUMAN]
Pterin-4-alpha-carbinolamine dehydratase OS = HS GN = PCBD1 PE = 1 SV = 2 - [PHS_HUMAN]
Profilin-1 OS = HS GN = PFN1 PE = 1 SV = 2 - [PROF1_HUMAN]
Profilin OS = HS GN = PFN2 PE = 1 SV = 1 - [C9J712_HUMAN]
Glycerol-3-phosphate phosphatase OS = HS GN = PGP PE = 1 SV = 1 - [PGP_HUMAN]
Fibrocystin-L OS = HS GN = PKHD1LI PE = 2 SV = 2 - [PKHL1_HUMAN]
Periostin OS = HS GN = POSTN PE = 1 SV = 1 - [B1ALD9_HUMAN]
Ribose-phosphate pyrophosphokinase 3 OS = HS GN = PRPS1L1 PE = 1 SV = 1 -
[A0A0B4J207_HUMAN]
Serine protease 23 (Fragment) OS = HS GN = PRSS23 PE = 1 SV = 1 - [E9PRR2_HUMAN]
Proteasome subunit alpha type-3 OS = HS GN = PSMA3 PE = 1 SV = 2 - [PSA3_HUMAN]
Proteasome subunit alpha type OS = HS GN = PSMA6 PE = 1 SV = 1 - [G3V295_HUMAN]
Proteasome subunit beta type-2 OS = HS GN = PSMB2 PE = 1 SV = 1 - [PSB2_HUMAN]
26S proteasome non-ATPase regulatory subunit 3 OS = HS GN = PSMD3 PE = 1 SV = 2 -
[PSMD3_HUMAN]
26S proteasome non-ATPase regulatory subunit 8 (Fragment) OS = HS GN = PSMD8 PE = 1 SV = 8 -
[K7EJR3_HUMAN]
Prostaglandin-H2 D-isomerase OS = HS GN = PTGDS PE = 1 SV = 1 - [PTGDS_HUMAN]
Peroxidasin homolog OS = HS GN = PXDN PE = 1 SV = 2 - [PXDN_HUMAN]
Sulfhydryl oxidase 1 OS = HS GN = QSOX1 PE = 1 SV = 3 - [QSOX1_HUMAN]
Ras-related protein Rab-11A (Fragment) OS = HS GN = RAB11A PE = 4 SV = 1 -
[H3BMH2_HUMAN]
Ras-related protein Rab-2B OS = HS GN = RAB2B PE = 1 SV = 1 - [E9PE37_HUMAN]
Ras-related protein Rab-5C (Fragment) OS = HS GN = RAB5C PE = 1 SV = 1 - [F8VVK3_HUMAN]
GTP-binding nuclear protein Ran (Fragment) OS = HS GN = RAN PE = 1 SV = 8 - [F5H018_HUMAN]
Retinoic acid receptor responder protein 2 OS = HS GN = RARRES2 PE = 1 SV = 1 - [RARR2_HUMAN]
60S acidic ribosomal protein P0 (Fragment) OS = HS GN = RPLPO PE = 1 SV = 1 - [F8VPE8_HUMAN]
40S ribosomal protein S2 (Fragment) OS = HS GN = RPS2 PE = 1 SV = 1 - [H0YEN5_HUMAN]
Ras suppressor protein 1 OS = HS GN = RSU1 PE = 1 SV = 3 - [RSU1_HUMAN]
Syndecan-4 OS = HS GN = SDC4 PE = 1 SV = 2 - [SDC4_HUMAN]
Alpha-1-antichymotrypsin OS = HS GN = SERPINA3 PE = 1 SV = 1 - [G3V3A0_HUMAN]
Plasminogen activator inhibitor 1 OS = HS GN = SERPINE1 PE = 1 SV = 1 - [PAI1_HUMAN]
Glia-derived nexin OS = HS GN = SERPINE2 PE = 1 SV = 1 - [GDN_HUMAN]
SH3 domain-binding glutamic acid-rich-like protein 3 OS = HS GN = SH3BGRL3 PE = 1 SV = 1 -
[Q5T123_HUMAN]
Sorbitol dehydrogenase OS = HS GN = SORD PE = 1 SV = 1 - [H0YLA4_HUMAN]
SPARC OS = HS GN = SPARC PE = 1 SV = 1 - [SPRC_HUMAN]
Testican-1 OS = HS GN = SPOCK1 PE = 1 SV = 1 - [TICN1_HUMAN]
Soluble scavenger receptor cysteine-rich domain-containing protein SSC5D OS = HS GN = SSC5D
PE = 1 SV = 3 - [SRCRL_HUMAN]
Stanniocalcin-2 (Fragment) OS = HS GN = STC2 PE = 1 SV = 1 - [HOYB13_HUMAN]
Tissue factor pathway inhibitor 2 OS = HS GN = TFPI2 PE = 1 SV = 1 - [TFPI2_HUMAN]
Thrombospondin-1 OS = HS GN = THBS1 PE = 1 SV = 2 - [TSP1_HUMAN]
Metalloproteinase inhibitor 1 OS = HS GN = TIMP1 PE = 1 SV = 1 - [TIMP1_HUMAN]
Metalloproteinase inhibitor 2 OS = HS GN = TIMP2 PE = 1 SV = 2 - [TIMP2_HUMAN]
Tenascin OS = HS GN = TNC PE = 1 SV = 3 - [TENA_HUMAN]
Tropomyosin alpha-3 chain OS = HS GN = TPM3 PE = 1 SV = 1 - [A0A087WWU8_HUMAN]
Tropomyosin alpha-4 chain OS = HS GN = TPM4 PE = 1 SV = 3 - [TPM4_HUMAN]
Translationally-controlled tumor protein OS = HS GN = TPT1 PE = 1 SV = 1 - [TCTP_HUMAN]
Translin (Fragment) OS = HS GN = TSN PE = 1 SV = 1 - [H7C1D4_HUMAN]
Tubulin beta chain OS = HS GN = TUBB PE = 1 SV = 1 - [Q5JP53_HUMAN]
Polyubiquitin-C (Fragment) OS = HS GN = UBC PE = 1 SV = 1 - [F5GYU3_HUMAN]
Ubiquitin-conjugating enzyme E2 N OS = HS GN = UBE2N PE = 1 SV = 1 - [F8VQQ8_HUMAN]
Versican core protein OS = HS GN = VCAN PE = 1 SV = 3 - [CSPG2_HUMAN]
Vimentin OS = HS GN = VIM PE = 1 SV = 1 - [B0YJC4_HUMAN]
Vacuolar protein sorting-associated protein 29 OS = HS GN = VPS29 PE = 1 SV = 1 -
[VPS29_HUMAN]

### Example 6: Immunogenicity & Immunomodulatory Properties of ASC

The expression of co-stimulatory molecules on ASC was measured. FACS analysis demonstrated the absence of CD80, CD86 and CD40 on the cell membranes. Moreover, the cells expressed low levels HLA class I molecules, as detected by staining for HLA A/B/C.

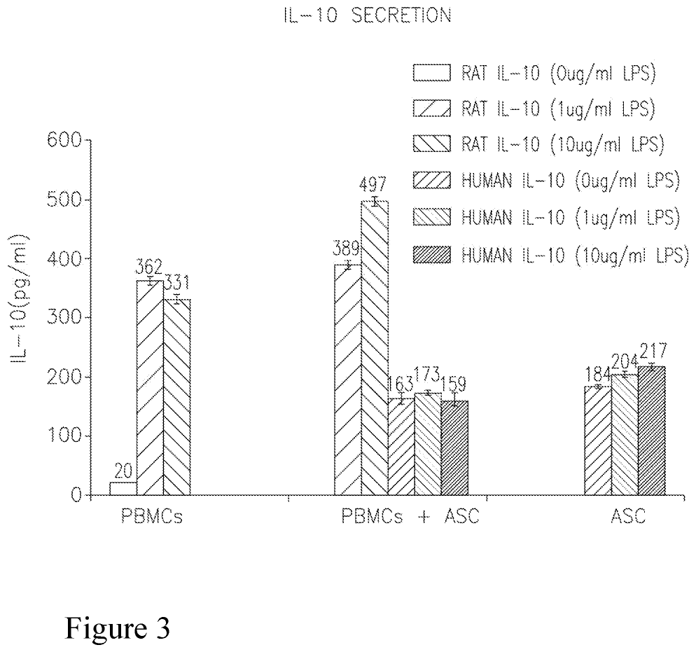

To further investigate the immunogenicity and the immunomodulatory properties of the cells, human/rat Mixed Lymphocyte Reaction (MLR) tests were performed. Rat PBMC were stimulated with LPS (lipopolysaccharide) in the absence or presence of (human) ASC, and secretion of IL-10 by the PBMC was measured. ASC increased the IL-10 secretion (FIG. 3).

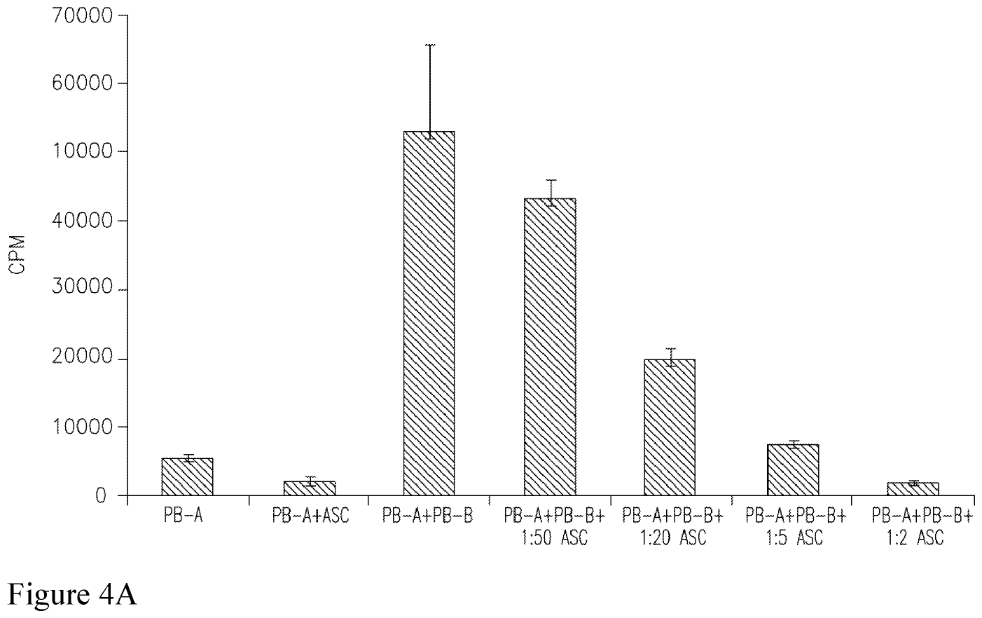

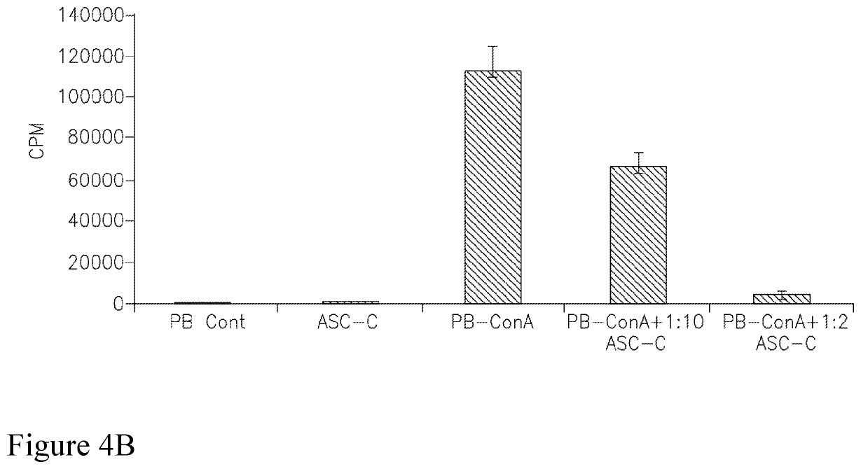

MLR performed with 2 different donors also showed that the ASC both escaped allorecognition (FIG. 4A) and reduced lymphocyte proliferation, as measured by thymidine incorporation, following mitogenic stimuli, such as Concanavalin A (Con A) (FIG. 4B) and Phytohemagglutinin (PHA; typically at least 25% inhibition relative to PHA alone), and non-specific stimulation by anti-CD3 and anti-CD28. The reduction in lymphocyte proliferation was dose dependent with the number of ASC.

Next, PBMC were stimulated by PHA using the Transwell® method (which prevents cell-to-cell contact but enables the diffusion of cytokines between the two compartments). The inhibition of proliferation was maintained even in this assay, showing that cell-to-cell contact was not necessary for the inhibition.

### Example 7: Adherent Stromal Cells Alter Cytokine Secretion by PBMC and Stimulate Endothelial Cell Proliferation

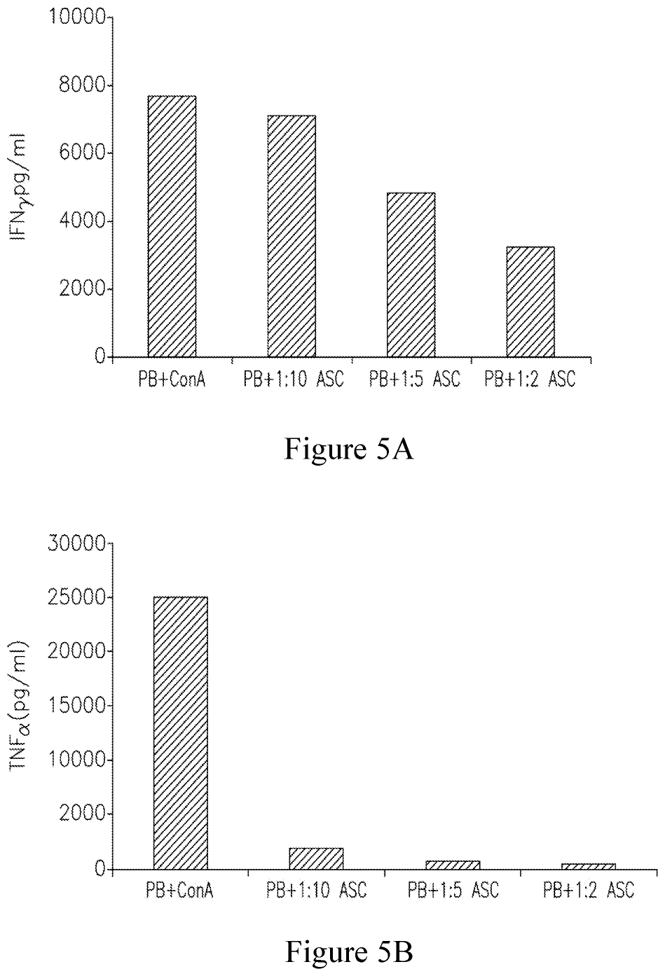

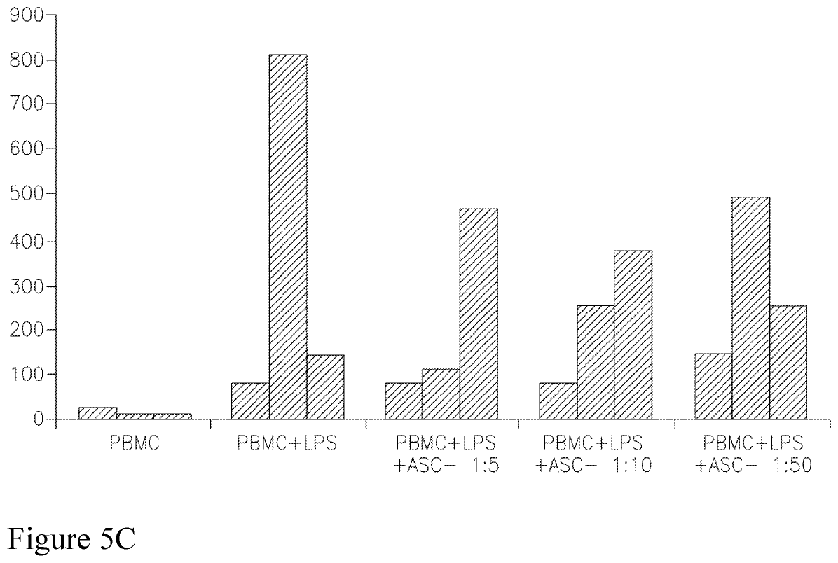

Additional co-culture studies were performed to test the effect of ASC on secretion of cytokines by lymphocytes. Culturing of PB-derived mononuclear cells (PBMC) with ASC slightly reduced IFN-gamma secretion and dramatically reduced TNF-alpha secretion by the PBMC, even when only low amounts of ASC were present (FIGS. 5A-B). Under conditions of LPS stimulation, the ASC increased secretion of IL-10 by PBMC, while decreasing their secretion of TNF-alpha, in a dose-dependent manner (FIG. 5C).

### Protocol—Endothelial Cell Proliferation (ECP) Assay:

ASC were prepared as described in Example 1, harvested by vibration, as described in PCT International Application Publ. No. WO 2012/140519, and were cryopreserved. 1×10⁶ thawed ASC were seeded in 2 ml DMEM medium. After 24 hours (hr), the medium was replaced with EBM-2 medium (Lonza Group Ltd, Basel, Switzerland), and cells were incubated under hypoxic conditions (1% O₂) for an additional 24 hr, after which the conditioned media was collected. In parallel, 750 human umbilical cord endothelial cells (HUVEC) were seeded, incubated for 24 hr, and then incubated with the conditioned media, for 4 days under normoxic conditions at 37° C. After removal of the conditioned medium, the proliferation of the HUVEC cells was assayed using the AlamarBlue® fluorescent assay. Results are presented as the percent ECP (%ECP) observed after PHA stimulation in the absence of ASC (arbitrarily set at 100%).

### Results

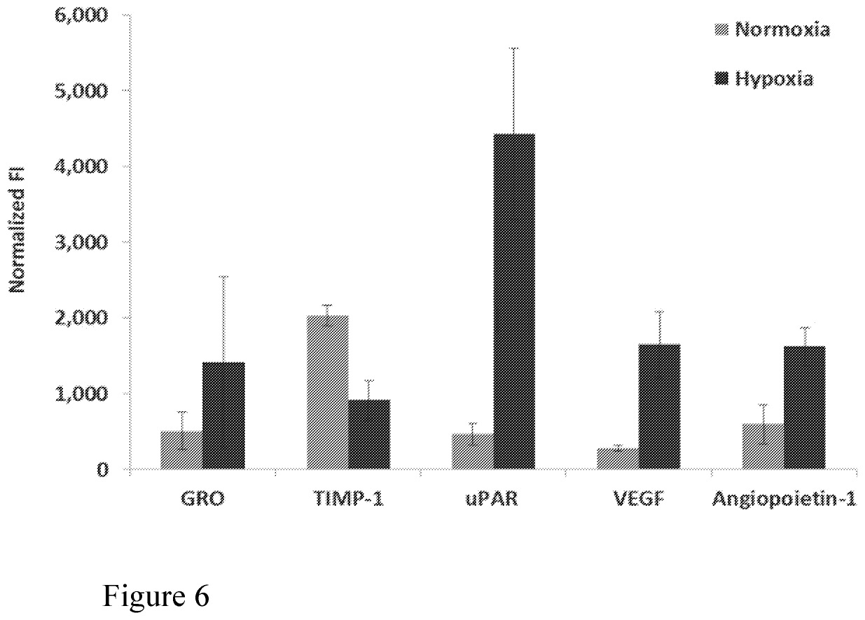

ASC cultured under normoxic or hypoxic conditions were tested for protein secretion, using Cytokine (Human) Antibody Array C Series 4000 (RayBio). Secretion of several pro-angiogenic factors was up-regulated under hypoxic conditions, as shown in FIG. 6. In additional experiments, various batches of ASC were co-incubated with HUVEC cells to test their effect on ECP. Stimulation of ECP was observed, typically at least 135% of ECP in the absence of ASC.

### Example 8: Cytokine Secretion by ASC Upon Exposure to Pro-Inflammatory Cytokines

Methods

General experimental protocol. ASC were obtained from the placenta and cultured under 2D conditions, then under 3D conditions, and were then harvested, all as described in Example 1, with the following deviation: One day before the end of the 3D culture (typically on day 5 or 6), the medium was replaced with DMEM, with or without the addition of 10 nanograms/milliliter (ng/ml) Tumor Necrosis Factor alpha (TNF-alpha), 10 ng/ml Interferon-Gamma (IFN-g), and/or 10% FBS (Table 4), and the bioreactor was incubated in batch mode (or, in selected experiments, in perfusion mode) for an additional day. Levels of secreted cytokines were measured in the bioreactor medium, using the RayBio® Human Cytokine Array kit.

Hypoxic incubation. 1×10⁶ thawed ASC were seeded in 2m1 DMEM medium. After 24 hours (hr), the medium was replaced with EBM-2 medium (Lonza Group Ltd, Basel, Switzerland), and cells were incubated under hypoxic conditions (1% O₂) for an additional 24 hr, after which the conditioned media was collected.

TABLE 4
Incubation conditions that were tested.

| Designation | Cytokines | FBS |
|---|---|---|
| 1 | None | NO |
| 2 | None | YES |
| 3 | TNF | NO |
| 4 | TNF | YES |
| 5 | TNF + IFN | NO |
| 6 | TNF + IFN | YES |

In other experiments, levels of secreted cytokines were measured in the conditioned medium (CM) from a hypoxic incubation, as described above.

Quantitative detection of secreted proteins: IL-6 was quantitatively measured using the human IL-6 immunoassay Quantikine® ELISA kit (R&D Systems). VEGF was quantitatively measured using the Human VEGF immunoassay Quantikine® kit (R&D Systems).

Results

In a series of experiments testing various conditions side-by-side, adherent stromal cells (ASC) were incubated in a bioreactor as described in the previous Examples. On the last day of the bioreactor incubation, the medium was replaced by medium containing or lacking added TNF-alpha and/or IFN-gamma, in the presence or absence of FBS. VEGF and IL-6 secretion were measured by ELISA. Inclusion of TNF-alpha significantly increased secretion of VEGF, whether or not IFN-gamma was present. In the same experiment, inclusion of TNF-alpha significantly increased IL-6 secretion, which was further increased by IFN-gamma

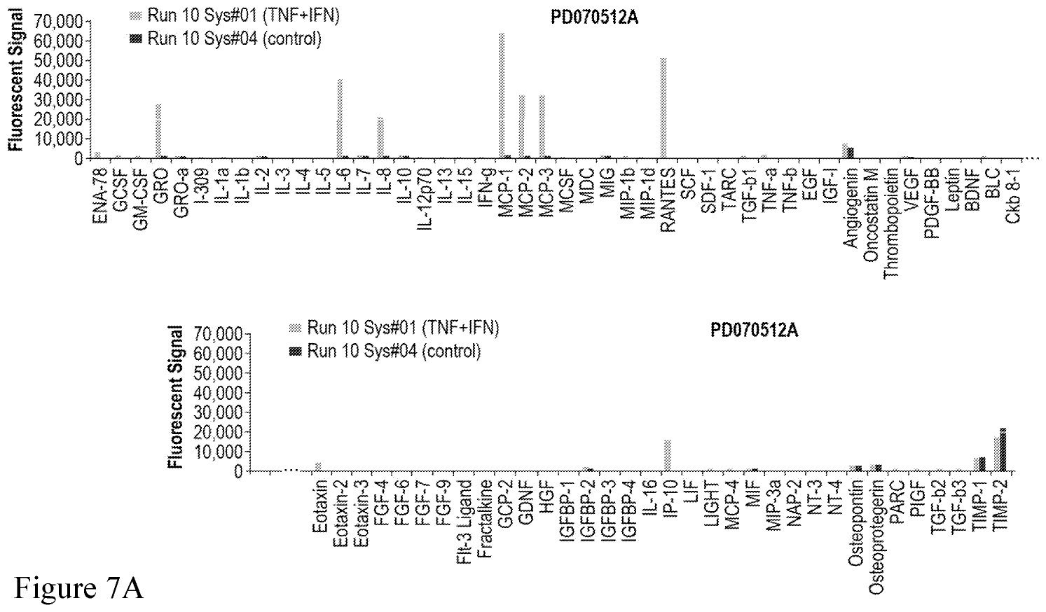

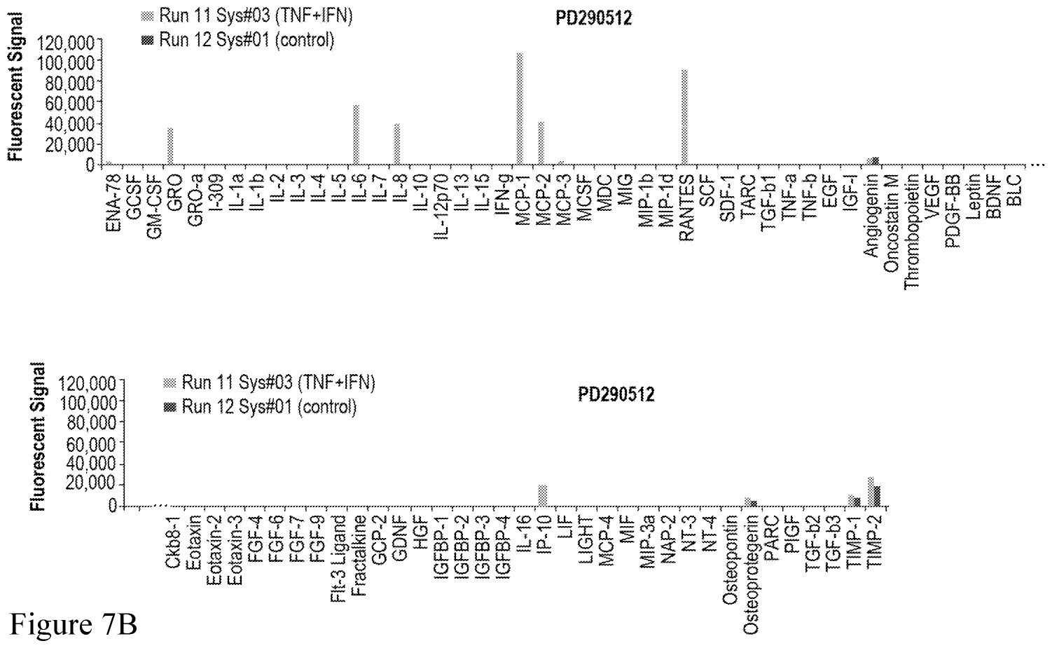

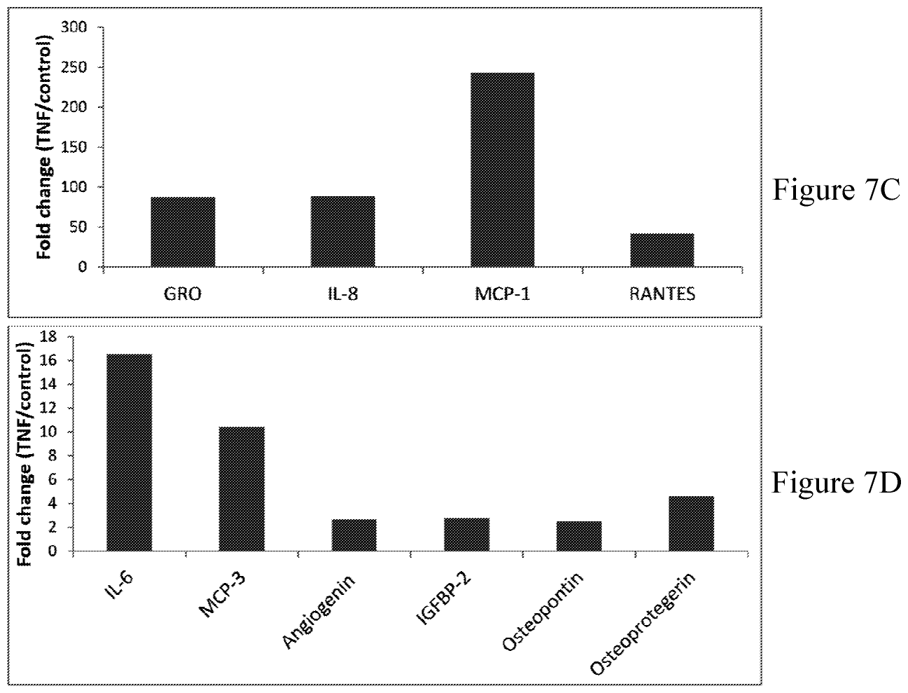

Expression of a panel of factors in the bioreactor media, all performed in the absence of serum, was measured by a fluorescence-based cytokine array assay, revealing the increased expression of several factors, including GRO, IL-6, IL-8, MCP-1, MCP-2, MCP-3, RANTES, and IP-10 (2 experiments are shown in FIGS. 7A-B). TNF-alpha stimulation was also compared to medium without cytokines, also in the absence of serum, showing increased expression of GRO, IL-8, MCP-1, RANTES, and, to a lesser extent, IL-6, MCP-3, Angiogenin, Insulin-like Growth Factor Binding Protein-2 (IGFBP-2), Osteopontin, and Osteoprotegerin (FIGS. 7C-D).

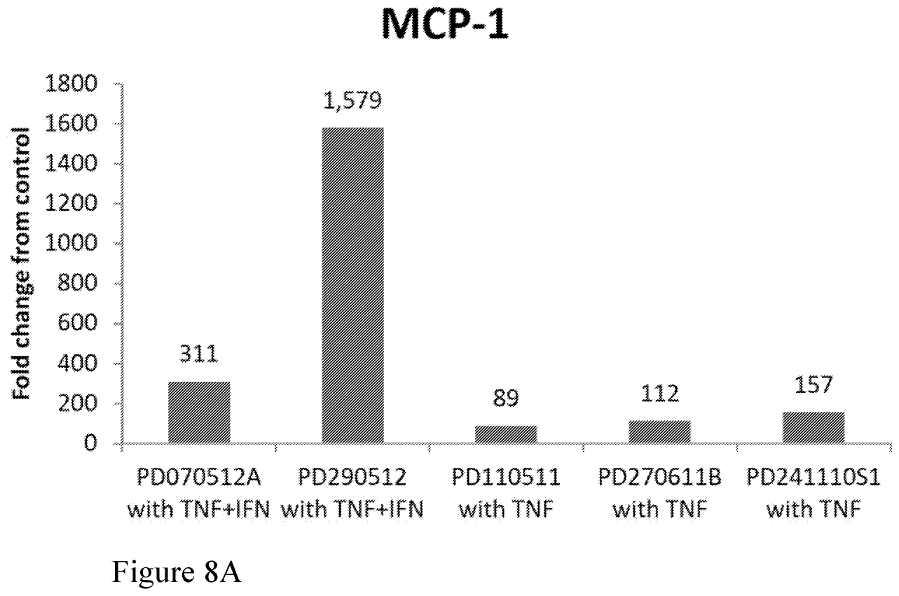

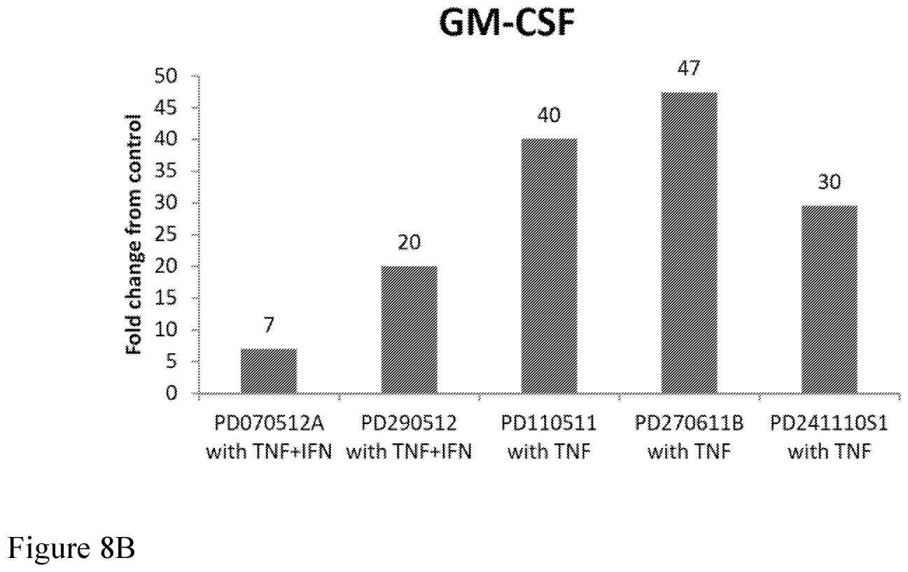

Increased expression of MCP-1 and GM-CSF in the bioreactor media was verified by quantitative ELISA in several experiments, all performed in the absence of serum. The results showed that TNF-alpha+IFN-gamma was superior to TNF-alpha alone for MCP-1 induction (FIG. 8A), while TNF-alpha alone appeared to be slightly superior for GM-CSF induction (FIG. 8B).

The next experiment examined the effect of FBS on induction of the aforementioned panel of factors by TNF-alpha+IFN-gamma or TNF-alpha alone. A similar set of major proteins, including RANTES, was induced in the presence or absence of FBS.

### Example 9: Quantitative Rantes ELISA on ASC

ASC were incubated with 10 ng/ml TNF-alpha, alone or in combination with 10 ng/ml IFN-gamma. The cells were cryopreserved, then thawed, and then 5×10⁵ cells were seeded in DMEM supplemented with 10% FBS and incubated under standard conditions. After 24 hours, the medium was replaced with 1-ml serum-free medium, and the cells were incubated another 24 hours under normoxic conditions. The medium was removed and assayed for RANTES secretion by ELISA, using the Quantikine® ELISA Human CCL5/RANTES kit (R&D Systems). The TNF-alpha+IFN-gamma-treated cells had sharply upregulated RANTES secretion compared to the other groups (Table 5).

TABLE 5
RANTES concentrations in culture medium.

| | | RANTES | Standard |
|---|---|---|---|
| Expt. No. | Conditions | conc. | dev. |
| 5 | No cytokines, no serum | 0 | 0 |
| 7 | No cytokines, serum. | 2 | 1 |
| 8 | No cytokines, serum | 0 | 0 |
| 5 | TNF-alpha, no serum | 76 | 2 |
| 7 | TNF-alpha, serum. | 591 | 20 |
| 8 | IFN-gamma + | 3232* | 83 |
| | TNF-alpha + serum. | | |

*Out of calibration curve.

### Example 10 Treatment of Pulmonary Arterial Hypertension With Placental ASC

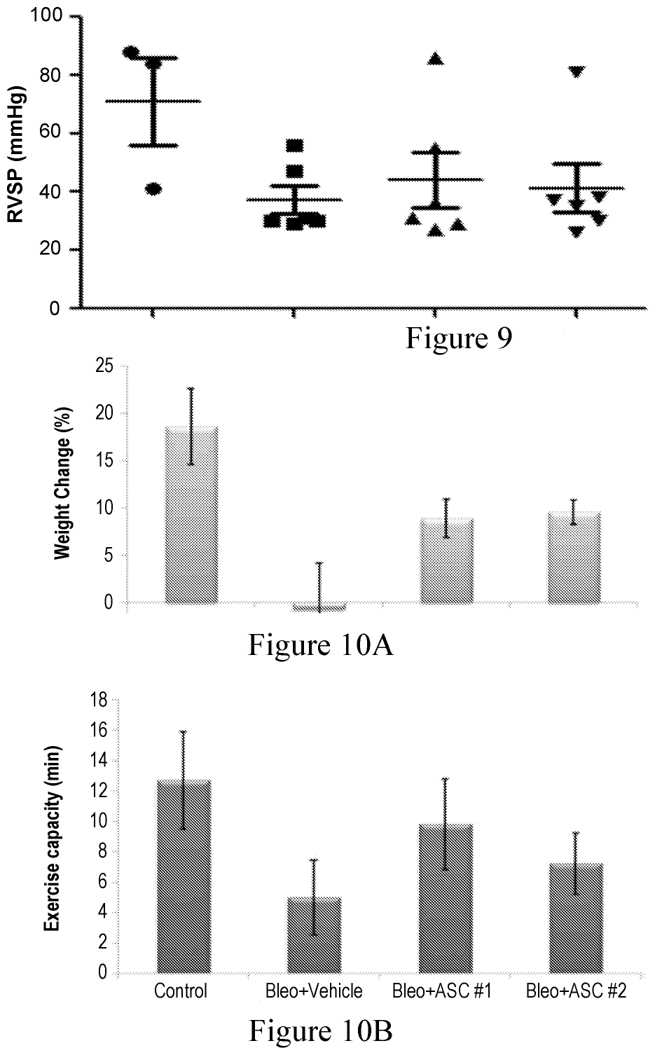

Nude rats were administered mock-treated or given monocrotaline (MCT) at 70 mg/kg intraperitoneally, and were administered intramuscularly 15, 25, or 35×10⁶/kg. placental/maternal ASC or vehicle on days 7 and 14. Right ventricular systolic pressure (RVSP) was assessed on day 24, and was found to be improved in the treated groups' lungs (FIG. 9). In another experiment, Sprague Dawley rats were mock-treated or given MCT, and were administered intravenously (slow bolus via tail vein) (1.33 or 6.67×10⁶/kg) either of 2 different batches of placental/maternal ASC or vehicle on days 7 or 14. RVSP on day 31 showed improvement in nearly all groups.

### Example 11: Treatment of Lung Fibrosis With Placental ASC

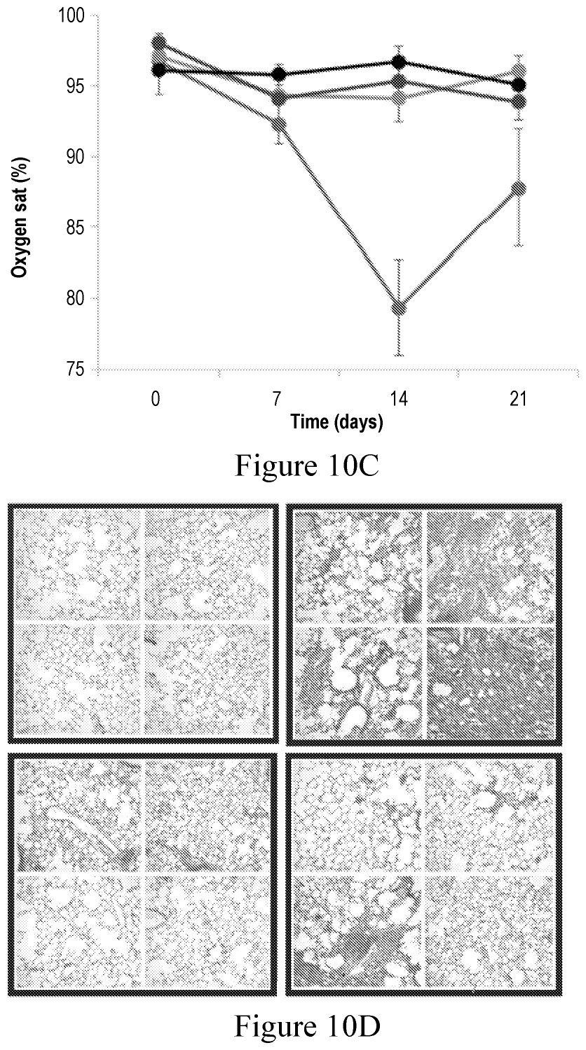

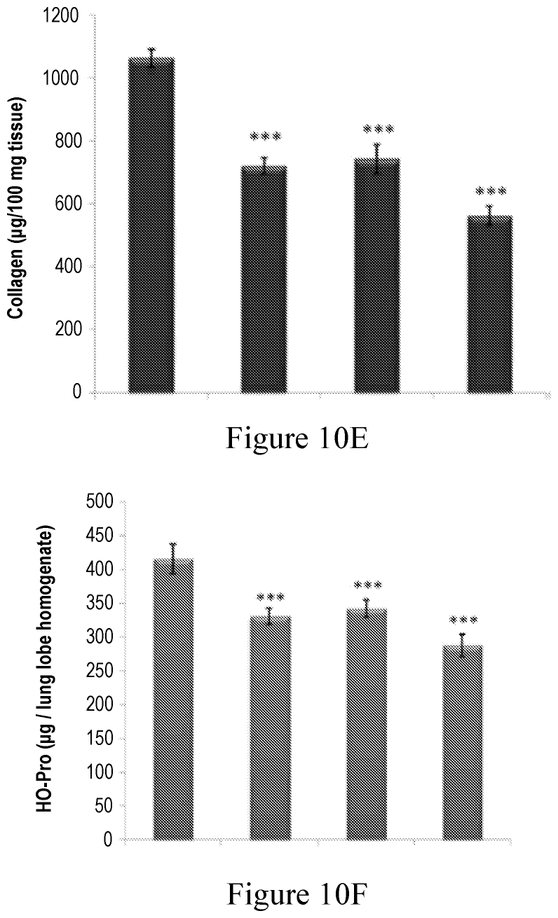

Adult male C57BL/6 mice (n=10/ group) were administered bleomycin intratracheally, followed by administration of 1 of 2 different batches of 0.5 million placental ASC, intratracheally. ASC treatment reduced weight loss, protected mice from loss in exercise capacity, prevented loss in O₂ saturation, drastically improved lung histology, and reduced collagen deposition in the lungs (FIGS. 10A-F).

### Example 12: Treatment of Acute Kidney Injury With Placental ASC

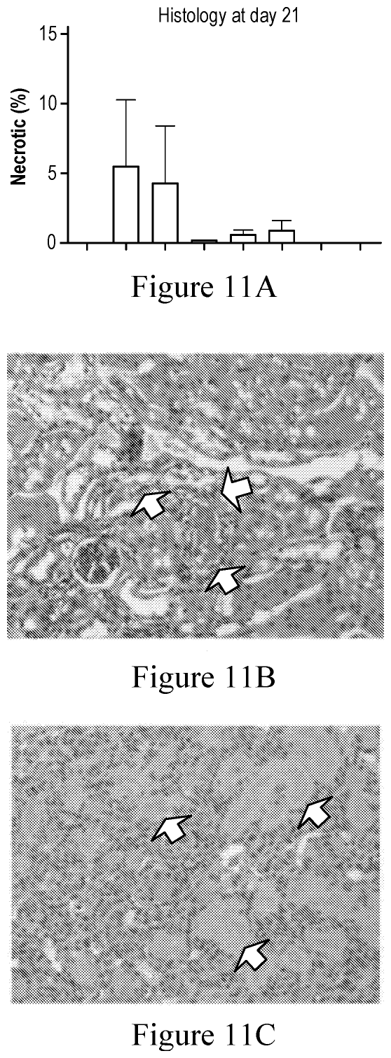

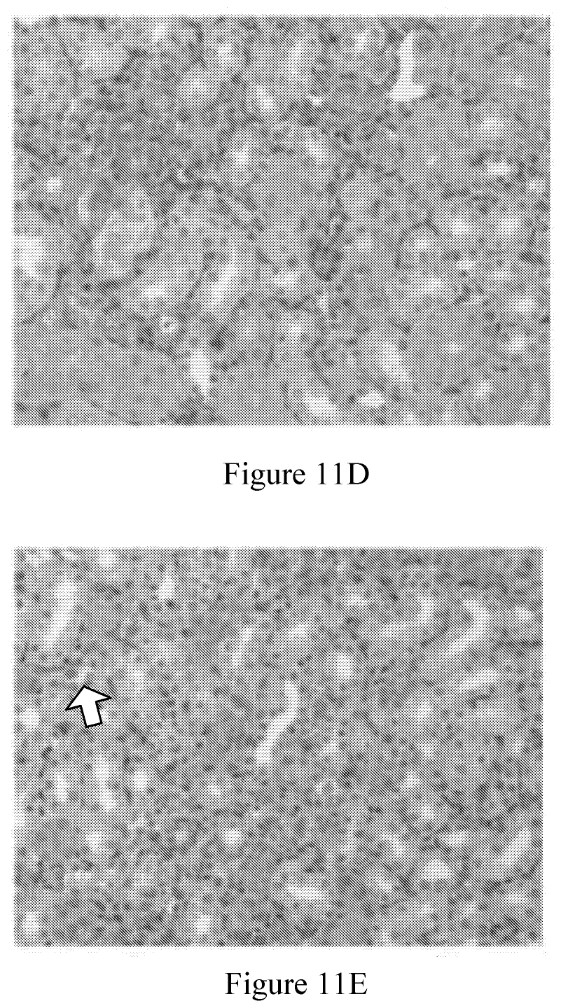

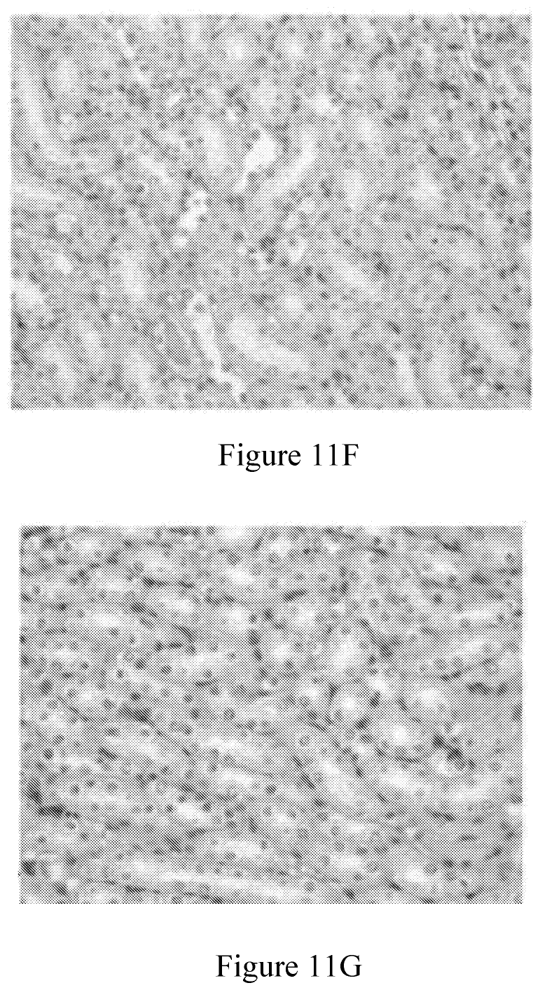

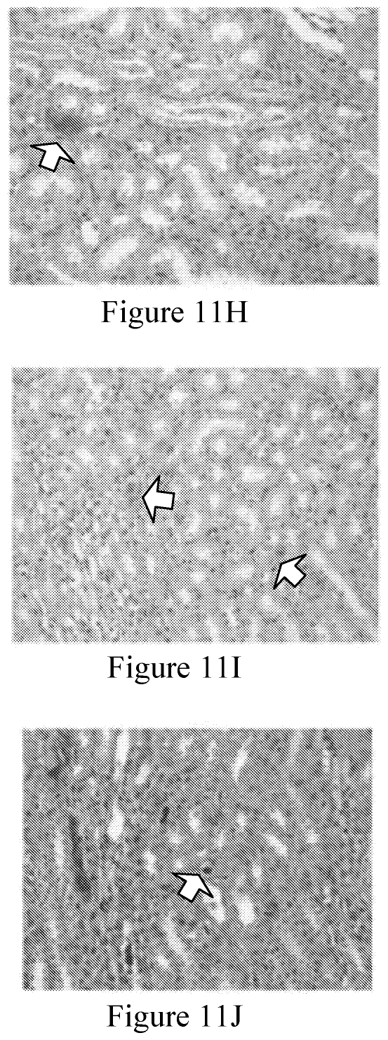

Male C57BL/6J mice aged 8-10 weeks were subjected to warm ischemia/reperfusion injury (IRI) by unilateral/right nephrectomy, followed by clamping the left renal artery and vein for one hour, then unclamping and reperfusion. 2.5 (low-dose) or 25 (high-dose) million/kg. placental ASC were administered IV or IM, 1 (=day 0), 24, or 72 hours after unclamping. There was a large decrease in percentage of necrotic tissue in the kidneys, as evidenced by histology (FIG. 11A). Images of representative day 21 Periodic Acid Schiff (PAS) tissue staining are shown for an untreated IRI mouse (B; 25.3% necrosis); low-dose IM ASC given on day 0 (C; 25.4% necrosis), 1 (D; 0.4% necrosis), or 3 (E; 2.3% necrosis); high-dose IM ASC given on day 1 (F; 0% necrosis) or 3 (G; 0% necrosis); and high-dose IV ASC given on day 0 (H; 4.5% necrosis), 1 (I; 4.5% necrosis), or 3 (J; 5.9% necrosis).

### Example 13: Treating Gastrointestinal Injury With Placental ASC

Male and female C57BL/6J mice were untreated (Group 1) or subjected to LD50/30 (1250 cGy) partial body irradiation (PBI) dose using 2.5% bone marrow sparing (Groups 2-4; n=30) as a model of gastrointestinal complications of viral pneumonia. 24 hours later, mice were administered vehicle (IM) (Group 2) or 2 doses of intramuscular (IM) placental/fetal ASC at a dose of 80 million cells/kg (or 80×10⁶ cells/kg) on days 1 and 3 (Group 3), or days 1 and 5 (Group 4). Statistically significant improvement for Day 30 survival was seen for combined sexes of Group 3 mice treated with ASC on Days 1 and 3 compared with control Group 2 mice treated with vehicle on Days 1 and 5 (Table 6).

TABLE 6
Survival of treated vs. untreated mice following GI injury.

| | | Treatment | | | Percent Survival | | | Total |
|---|---|---|---|---|---|---|---|---|
| | | Regimen | No. Survived to Day 30 | | Male | Female | Both | Percent |
| Group | Treatment | (Days) | Males | Females | alone | alone | sexes | Lethality |
| 2 | Vehicle Control | 1 and 5 | 5 | 2 | 21% | 8% | 15% | 85% |
| | Plasma-Lyte 148 | | | | | | | |
| 3 | ASC | 1 and 3 | 10 | 4 | 42% | 17% | 29% | 71% |

### Example 14: Treatment of Viral-Induced Pneumonia and Complications With Placental ASC

Methods

Data Analysis

All data analyses were conducted in version 3.6.1 of R (R Core Team, 2019). Using the lmerTest package, baseline adjusted repeated measures linear models were fit with restricted maximum likelihood (REML), and t-tests were computed using Satterthwaite's method. Time points in which only a single subject contributed data were omitted before running the repeated measures models.

Patient Characteristics

The general characterization of the patients is described in Table 7. Eight patients (7 males and 1 female) were treated. All patients were confirmed for SARS-CoV-2 infection by real-time reverse transcriptase polymerase chain reaction (RT-PCR). The median age of the patients was 55 years (range 22-79 years). 5/8 patients were at higher risk for severe illness from COVID-19 due to underlying medical conditions. The most common comorbidities were hypertension (4 patients) and diabetes (4 patients), with 3 patients suffering from both. 7 patients had BMI above 25. None of the patients were active smokers. Prior to ASC treatment, all patients had received hydroxychloroquine, 3 patients received lopinavir/ritonavir, and 2 patients received remdesivir. In addition, 3 patients had received IL-6 inhibitors, and 4 patients had received steroids. Two patients needed Extracorporeal Membrane Oxygenation (ECMO). The first, patient #6, needed resuscitation due to a massive pulmonary embolism, occurring 4 days after ASC treatment, and was placed on ECMO until full recovery. The second, #4, was electively placed on ECMO due to severe non-resolving ARDS and hypoxia. The average length of hospital stay was 41 days for the six patients being discharged by follow-up end. 5/8 patients were intubated for at least 5 days before ASC treatment, with 1 patient (#8) being intubated for 22 days prior to ASC treatment.

TABLE 7
General characteristics of patient population.

| Subject # | 1 | 2 | 3 | 4 | 5 | 6 | 7 | 8 |
|---|---|---|---|---|---|---|---|---|
| Site | Rambam | Bnai | Rambam | Assuta | Assuta | Assuta | Bnai | Holy |
| | | Zion | | | | | Zion | Cross |
| Age | 71 | 79 | 56 | 54 | 53 | 22 | 65 | 49 |
| Sex | M | M | M | M | M | M | F | M |
| BMI | >30 | >30 | 29.5 | 24.4 | 30 | 30.5 | >30 | 27.8 |
| Active smoker | N | N | N | N | N | N | N | N |
| Diabetes | Y | N | Y | N | Y | N | Y | N |
| Hypertension | Y | Y | Y | N | N | N | Y | N |
| COPD | N | N | N | N | N | N | N | N |
| Ischemic heart | N | N | N | N | N | N | N | N |
| disease | | | | | | | | |
| Number ASC | 2 | 1 | 2 | 1 | 1 | 1 | 2 | 1 |
| treatments | | | | | | | | |
| Other investigat. | H, L | H, R | H, L | H, IL6 | H, IL6 | H | H, L, | H, R |
| drugs * | | | | | | | IL6 | |
| Steroids | Y | Y | N | N | N | Y | N | Y |
| No. hospital days | 26 | 69 | 22 | NA | 27 | 48 | NA | 56 |
| Days intubated | 5 | 14 | 1 | 10 | 10 | 2 | 2 | 22 |
| before treatment | | | | | | | | |
| Days intubated | 20 | 16 | 7 | ongoing | 14 | NA | 35 | 11 |
| after treatment | | | | | | | | |
| Status | Died | Dis'd* | Dis'd | In hosp. | Dis'd | Dis'd | In hosp. | Dis'd |

* H = hydroxychloroquine; L = Lopinavir/ritonavir; R = Resmdesivir; IL6 = anti IL-6;
NA = data not available.
Dis'd = discharged.

Results

Eight 2019-nCoV-infected patients, all in Intensive Care Units (ICU) on invasive mechanical ventilation and suffering from Acute Respiratory Distress Syndrome (ARDS), were enrolled in a compassionate use program over a 9-day period. Patients were administered 300 million maternal placental ASC in a mixture containing 10% DMSO, 5% human serum albumin and Plasma-Lyte®, via 15 IM injections (1 mL each). 3/8 received an additional treatment of 300 million cells 8 or 11 days later, according to the physician's discretion.

COVID-19-infected patients that require invasive mechanical ventilation are considered at high risk for mortality (Bhatraju P K et al.). Despite that, the patients studied exhibited a 100% survival rate at the first assessment, which was at least 1 week after treatment for 6/7 of the patients and 2 days after treatment for the 7^{th}. Furthermore, 4 patients exhibited multi-organ failure prior to treatment; 2/4 (50%) exhibited clinical recovery in addition to the respiratory improvement.

No adverse events attributed to ASC treatment were reported.

Improvement in CRP Levels

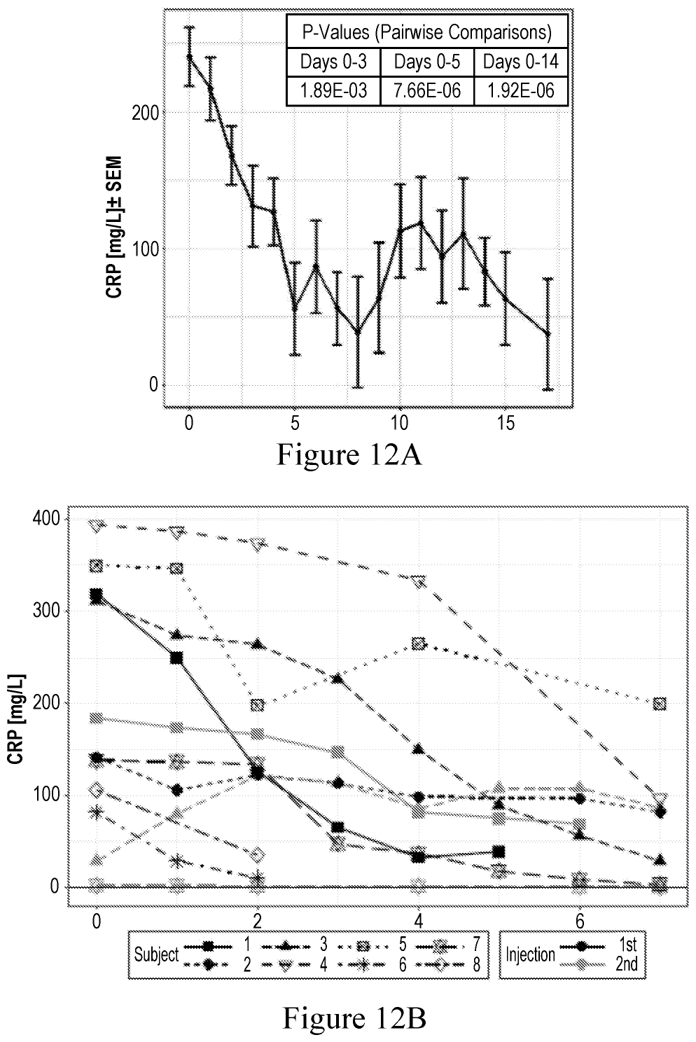

By day 3 post injection, mean CRP had fallen 45% (240.3 mg/L to 131.3 mg/L, p=1.9×10⁻³) and by day 5, 77% (to 56.0 mg/L, p=7.7×10⁻⁶) (FIG. 12A). The drop in blood CRP following injection of ASC either on day 0 (data from all subjects is shown) or, if the subject had a second injection, on either day 8 or 11 (day 11 for subject #1 or day 8 for subjects #3 and #7) is shown in FIG. 12B. These data generally show that the higher the level of blood CRP, the more dramatic the reduction following treatment.

Improvement in Respiratory Parameters

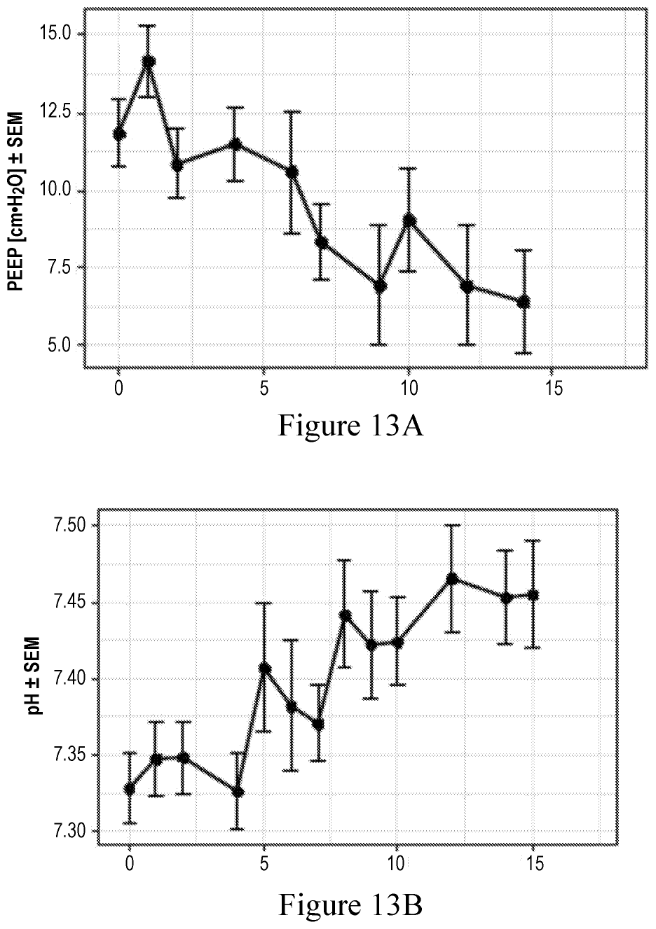

PaO_{2/}FiO₂ increased in 5/8 patients 24 h post treatment, with a similar effect 48 h post treatment (Table 8). A decrease in PEEP (Positive End Expiratory Pressure) and increase in pH were both statistically significant between days 0-14 (p=3.2×10⁻³ and p=7.2×10⁻⁴, respectively; FIGS. 13A-B).

TABLE 8
P/F ratio of patients before and after treatment.

| Patient | Before | 24 h Post | 48 h Post |
|---|---|---|---|
| No. | Treatment | Treatment | Treatment |
| 1 | 160 | 229 | 170 |
| 2 | 140 | 172.5 | 177.5 |
| 3 | 143 | 151 | 217 |
| 4 | 149 | 107 | NA |
| 5 | 106 | 145 | NA |
| 6 | 173 | 205 | NA |
| 7 | 172 | 93 | 95 |
| 8 | 342.5 | NA | 425 |

NA = data not available

Most of the patients (6 out of 8) required vasopressor treatments prior to enrollment. In 3 patients, vasopressor doses could be decreased following ASC treatments and/or discontinued as early as 3 days post treatment (patient #3).

Changes in Chest Radiographs

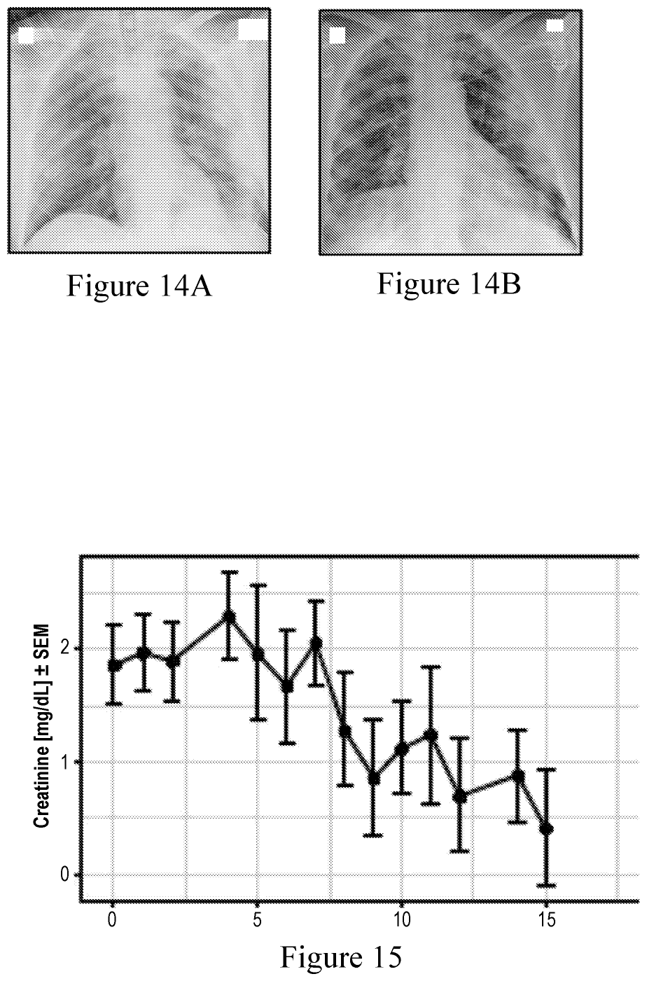

Chest radiographs were obtained from 6 patients. In patients #1 and #3, the radiographs demonstrated some resolution and improvement in the interstitial opacities. FIG. 14 provides the image from patient #3. Some improvement was also seen in patients #5, #6, and #8. No improvement was seen in patient #4. (Data prior to ASC treatment was not available for patients #2 and #7).

Improvement in Kidney Function

A decrease in creatinine, indicative of acute kidney injury, was statistically significant between days 0-14 (p=3.2×10⁻²; FIG. 15), representing a 53% drop (from 1.875 to 0.883 mg/dL).

28-day Follow Up Data

After a 28-day follow-up period, 7/8 (87.5%) of the subjects were still alive, 6/8 (75%) had been weaned off mechanical ventilation, and 5/8 (62.5%) had been discharged alive from the hospital. By contrast, mortality rates of ARDS COVID-19 patients who require mechanical ventilation are reported to range between 24.5% to 94% in different studies (Richardson S et al. and Gibson P G et al.)

### Example 15: Further Testing of Placental ASC for Treatment of Viral-Induced Pneumonia and Complications Thereof

High-risk 2019-nCoV-infected patients are administered placebo (Plasma-Lyte®), (negative control); or 300 or 600 million maternal placental ASC, via 15 or 30 IM injections, in a clinical trial. Some groups receive 2 doses of placebo or 300 (or 600) million ASC, spaced one week apart. Amelioration of pneumonia, complications thereof, or other sequelae of viral infection is indicative of therapeutic efficacy.

### Example 16: Use of ASC in Treating Viral-Induced Sequelae

Recovered 2019-nCoV-infected patients complaining of continuing symptoms (fatigue; anxiety; shortness of breath; sustained cough; or limb pain [e.g., arm and/or leg pain], such as a burning sensation, a tingling, or a feeling of unease) are administered placebo (Plasma-Lyte®), (negative control); or 300 or 600 million maternal placental ASC, via 15 or 30 IM injections, in a clinical trial. Some groups receive 2 doses of placebo or 300 or 600 million ASC, spaced one week apart. Amelioration of continuing symptoms is indicative of therapeutic efficacy.

### Example 17: Use of ASC in Treating an Animal Model of ARDS

Methods

Female BALB/c mice were challenged with intratracheal instillation of LPS, followed by intramuscular administration of placental ASC (2 different batches) or placebo group. A naive (unchallenged) group served as an additional control. 72 h after LPS challenge, bronchoalveolar lavage was performed, and mice were sacrificed. Cytokines were measured in lavage fluid (BALF) and serum, and histopathology was performed on the lung specimens. Lung injury score is a composite of scores (0-2 each) for neutrophil infiltration, fibrin deposition, and alveolar thickening. For inter-group comparisons, ANOVA was performed, followed by Tukey's multiple comparisons test.

Results

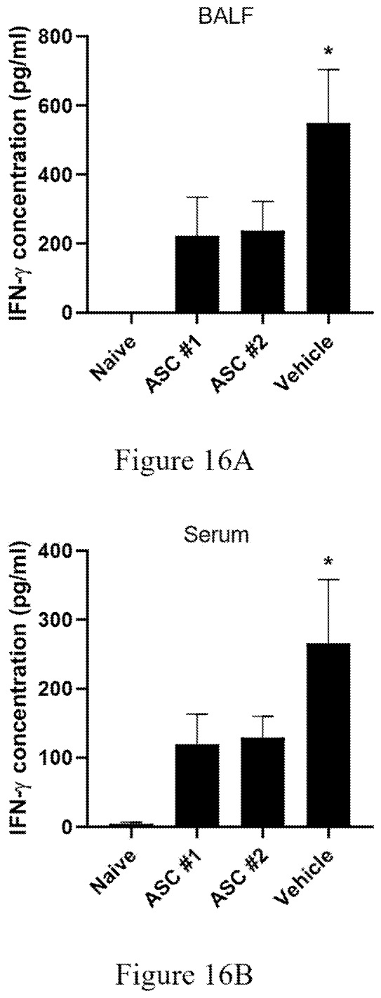

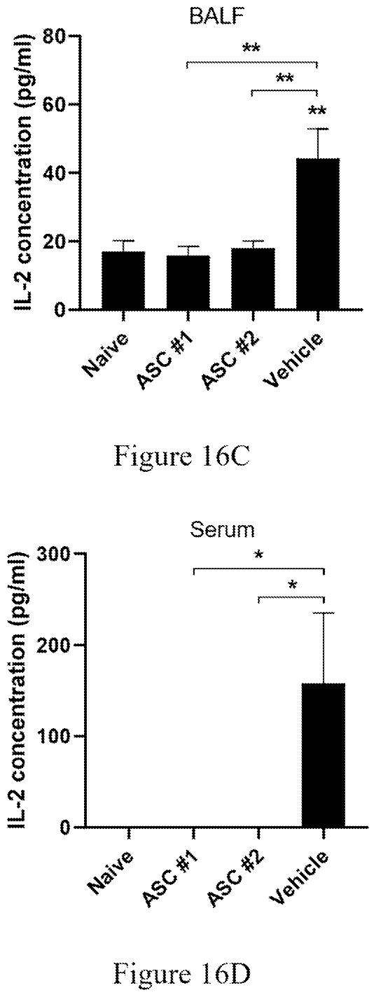

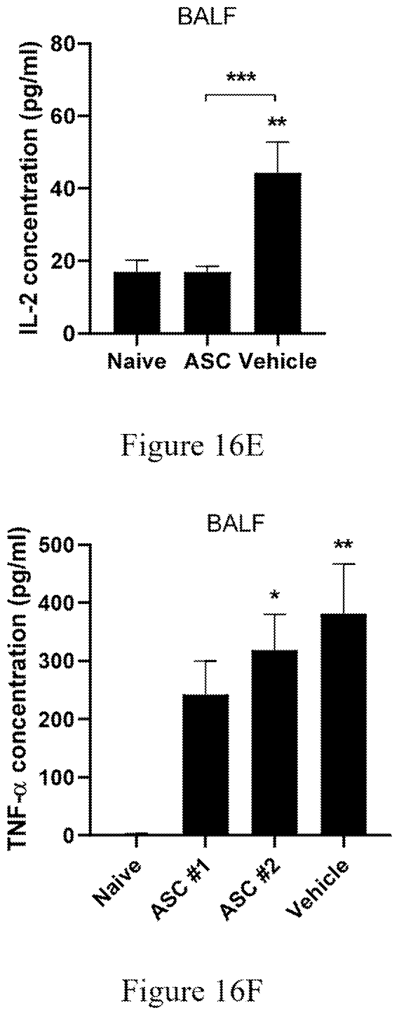

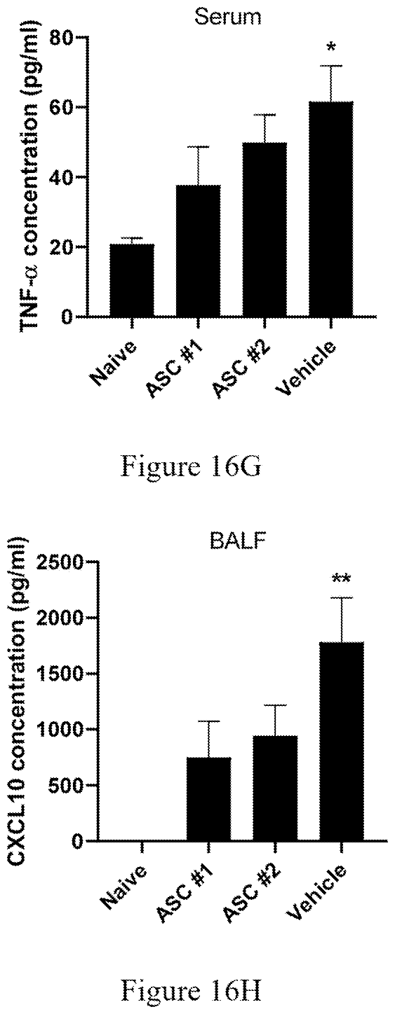

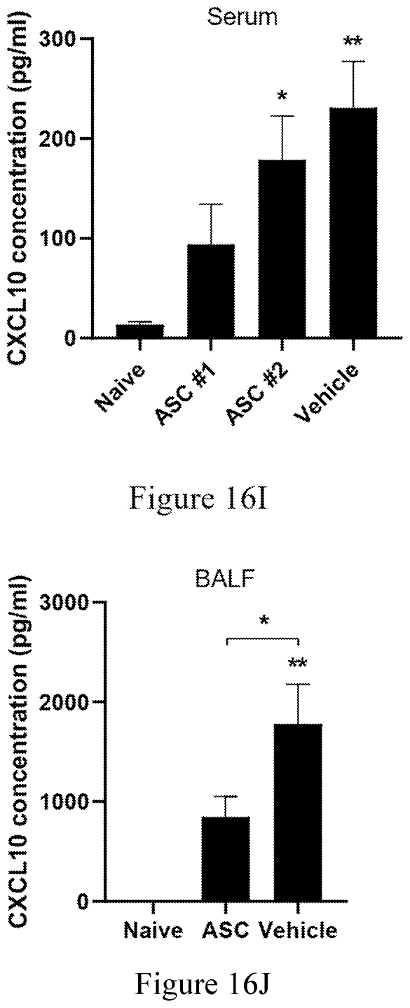

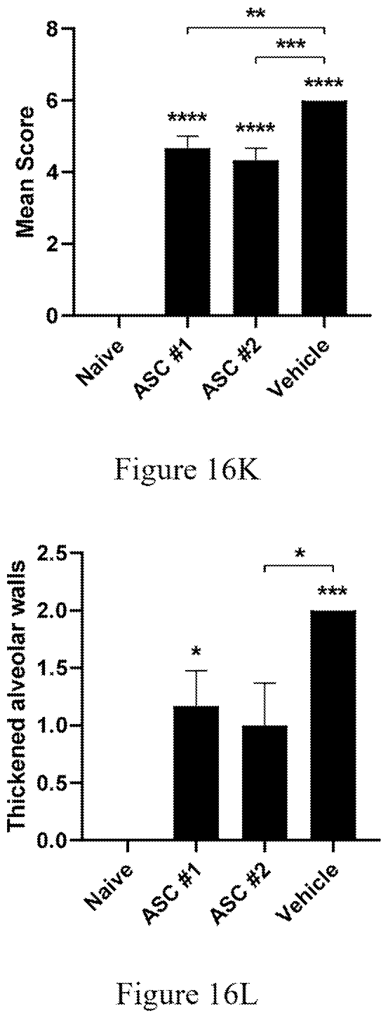

ASC treatment showed a clear trend of reduced levels of several hyperinflammatory markers (Chaudhari S et al.) in both BALF and serum, for example IFN-g (FIGS. 16A-B), interleukin 2 (IL-2) (C-E), TNF-a (F-G), and CXCL-10 ((IP-10; Uniprot Accession No. P02778; accessed on Mar. 10, 2021) (H-J). In E and J, both ASC-treated groups were combined into the single dataset. Furthermore, lung injury score and alveolar thickening (K-L) were both significantly reduced in the treated groups.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### References (Additional References May Be Cited in Text)

Bhatraju P K et al., Covid-19 in Critically Ill Patients in the Seattle Region-Case Series. N Engl J Med. 2020 Mar. 30. doi: 10.1056/NEJMoa2004500. [Epub ahead of print]
Bray M et al., Haematological, biochemical and coagulation changes in mice, guinea-pigs and monkeys infected with a mouse-adapted variant of Ebola Zaire virus. J Comp Pathol. 2001 November;125(4):243-53.
Chaudhari S et al., Comorbidities and inflammation associated with ovarian cancer and its influence on SARS-CoV-2 infection, J Ovarian Res. 2021 Feb. 25;14(1):39.
Dominici et al., Minimal criteria for defining multipotent mesenchymal stromal cells. The International Society for Cellular Therapy position statement. Cytotherapy. 2006; 8(4):315-7.
Geisbert T W et al., Mechanisms underlying coagulation abnormalities in ebola hemorrhagic fever: overexpression of tissue factor in primate monocytes/macrophages is a key event. J Infect Dis. 2003 Dec. 1;188(11):1618-29.
Gibson P G et al., COVID-19 ARDS: clinical features and differences to “usual” pre-COVID ARDS. The Medical Journal of Australia. 2020. [Preprint, 24 Apr. 2020].
Kinzebach S and Bieback K. Expansion of Mesenchymal Stem/Stromal cells under xenogenic-free culture conditions. Adv Biochem Eng Biotechnol. 2013;129:33-57.
Monaca E et al, Assessment of hemostaseologic alterations induced by hyperbaric oxygen therapy using point-of-care analyzers. Undersea Hyperb Med. 2014 January-February;41(1):17-26.
Richardson S et al., Presenting Characteristics, Comorbidities, and Outcomes Among 5700 Patients Hospitalized With COVID-19 in the New York City Area. JAMA 2020. doi:10.1001/jama.2020.6775
Yen J Y et al., Therapeutics of Ebola hemorrhagic fever: whole-genome transcriptional analysis of successful disease mitigation. J Infect Dis. 2011 November;204 Suppl 3:S1043-52.

1. A method for treating or ameliorating a coronavirus infection, comprising administering a composition that comprises a cultured placental adherent stromal cell (ASC), thereby treating or ameliorating a coronavirus infection.

2. A method for treating or ameliorating a complication of a coronavirus infection, comprising administering a composition that comprises a cultured placental adherent stromal cell (ASC), thereby treating or ameliorating a complication of a coronavirus infection.

3. A method for treating, preventing, or ameliorating a pneumonia, comprising administering a composition that comprises a cultured placental adherent stromal cell (ASC), wherein said pneumonia is associated with a viral infection, thereby treating, preventing, or ameliorating pneumonia.

4. A method for treating, preventing, or ameliorating a complication of a pneumonia, comprising administering a composition that comprises a cultured placental adherent stromal cell (ASC), wherein said pneumonia is associated with a viral infection, thereby treating, preventing, or ameliorating a complication of pneumonia.

5-8. (canceled)

9. The method of claim 1, where said composition is an injected composition.

10. (canceled)

11. The method of claim 1, wherein said placental ASC have been incubated on a 3D substrate.

12. The method of claim 11, wherein said placental ASC have been incubated on a 2D substrate, prior to incubating on a 3D substrate.

13. The method or composition of claim 11, wherein said 3D culture substrate comprises a synthetic adherent material, wherein said synthetic adherent material is a fibrous matrix.

14. (canceled)

15. The method of claim 13, wherein said synthetic adherent material is selected from the group consisting of a polyester, a polypropylene, a polyalkylene, a polyfluorochloroethylene, a polyvinyl chloride, a polystyrene, and a polysulfone.

16. The method of claim 11, wherein said 3D culture apparatus comprises microcarriers disposed within a bioreactor.

17. The method of claim 1, wherein said placental ASC is allogeneic to said subject.

18. The method of claim 1, wherein the composition is intramuscularly injected.

19. The method of claim 1, comprising 100-600 million of said placental ASC, for an adult subject.

20. The method of claim 1, wherein said composition comprises:
a. a first pharmaceutical composition, comprising allogeneic placental ASC from a first donor; and
b. a second pharmaceutical composition, comprising allogeneic placental ASC from a second donor, wherein said second donor differs from said first donor in at least one allele group of human leukocyte antigen (HLA)-A or human leukocyte antigen (HLA)-B.

21. (canceled)

22. The method of claim 4, wherein said complication is lung fibrosis.

23. The method of claim 2, wherein said complication is systemic inflammatory response syndrome.

24. The method of claim 1, wherein said ASC express a marker selected from the group consisting of CD73, CD90, CD29 and CD105.

25. The method of claim 1, wherein said ASC do not express a marker selected from the group consisting of CD3, CD4, CD11b, CD14, CD19, and CD34.

26. The method of claim 1, wherein said ASC do not express a marker selected from the group consisting of CD3, CD4, CD34, CD39, and CD106.

27. The method of claim 26, wherein less than 50% of said ASC express CD200.
